(19) United States
(12) Patent Application Publication
(10) Pub. No.: US 2026/0256691 A1
(43) Pub. Date: Sep. 3, 2026

(54) FILM DELIVERY SYSTEM WITH PERFORATIONS

(71) Applicant: LTS LOHMANN THERAPIE-SYSTEME AG, Andernach (DE)

(72) Inventor: Stefan LORSCHEIDT, Bonn (DE)

(21) Appl. No.: 19/162,573

(22) PCT Filed: Mar. 7, 2024
(86) PCT No.: PCT/EP2024/056016

§ 371 Date: Sep. 5, 2025

(30) Foreign Application Priority Data
Mar. 7, 2023 (EP) 23160597.3

(51) Int. Cl. A61K 9/00; A61K 47/10; A61K 47/26; A61K 47/32; A61K 47/38

(52) U.S. Cl. CPC A61K 9/006; A61K 47/10; A61K 47/26; A61K 47/32; A61K 47/38

(57) ABSTRACT

The present invention relates to film delivery systems comprising a mucoadhesive layer, wherein the mucoadhesive layer comprises a film-forming agent and is provided with perforations.

### TECHNICAL FIELD OF THE INVENTION

The present invention relates to a film delivery system comprising a mucoadhesive layer provided with perforations, and processes of manufacture, methods of treatment and uses thereof.

### BACKGROUND OF THE INVENTION

Drug delivery by conventional oral dosage forms such as tablets, pills, caplets, and capsules is a standard route of drug administration. However, for the elderly and the infants, tablets and the like may be difficult to swallow. In addition, peroral administration is associated with pre-systemic elimination of the active agent, which may include gastrointestinal (GI) degradation, metabolism, or first-pass clearance via the liver, and often suffers from limitations such as low systematic bioavailability, food effect, slow onset of action, and/or formation of inactive, and at times toxic metabolites.

In view of such limitations, dosage forms which may be in the form of a thin film such as transmucosal therapeutic systems have gained relevance in the past years. Oromucosal drug delivery offers the advantage of combining the ease and convenience of use of oral administration with bypassing the GI-tract, since the drug is absorbed into the systemic circulation directly via the mucosal tissue. When compared to oromucosal dosage forms such as orodispersible tablets, oromucosal film delivery systems are advantageous in causing less discomfort to the patient, since they can be applied on the mucosa and then ideally will be no more or barely perceived by the patient.

Transmucosal therapeutic systems, or transmucosal delivery systems in the form of a film consist of one or more thin layers which are applied and adhere to the mucosa, e.g., of the oral cavity, to deliver the drug over a period of time. In such a transmucosal therapeutic system, the active is contained in a dissolvable layer, and active delivery is intended to be achieved at least partly through the mucosa to which the film adheres.

Dosage forms in the form of thin films for application in the oral cavity (including transmucosal therapeutic systems) are also sometimes referred to as “Oral Thin Film” or OTF, however, OTFs are not necessarily intended to adhere to the mucosa. Such OTFs may also be intended to disintegrate fast in the saliva (referred to e.g. as “flash wafers”) and e.g. thus offer the advantage to aid the patient taking the medication.

In summary, as outlined above, dosage forms in the form of a film, i.e., film delivery systems, offer various alternative drug delivery forms and administration routes which may be advantageous in terms of convenience, ease and comfort for the patient, and/or by offering improved pharmacokinetic behavior such as higher bioavailability and faster onset of action. In more detail, because of their thin and flexible nature, films are less obtrusive and more acceptable by the patient when compared, e.g., to tablets. When formulated e.g. as (trans)mucosal administration forms, film delivery systems can deliver the active directly to the systemic circulation avoiding the gastrointestinal (GI-) tract, which may improve the drug efficacy and also eliminate side effect due to first-pass metabolism, and thus present a non-invasive administration form which is a promising alternative to conventional oral dosage forms.

However, film delivery systems are a relatively new form of drug delivery, meaning that knowledge on formulation technology is limited. Formulating appropriate film delivery systems, e.g., for the transmucosal delivery is challenging due to a multitude of aspects to be considered and issues to be solved. The main requirements for such film delivery systems are good adhesion and active permeation, combined with an appropriate behavior and time of disintegration. Since the application of thin films should not cause discomfort to the patient, the haptics of the films needs to be taken into account as a basic physical quality.

The design of thin films requires unique consideration of dimension and thickness of the film and drug to polymer ratio as the films need to be thin enough to not be perceived by the patient. This requirement limits the thickness and thus the drug loading capacity of the films. Higher doses would increase the thickness of the films which would result in increased dissolution times and/or increased foreign body sensation, which renders the delivery system less convenient for patients. In addition, film-forming materials which form relatively rigid films might be desirable in view of dissolution properties and/or drug release behavior, but are limited in use since they will cause more easily a foreign-body sensation. Other film-forming agents might be desirable in terms of providing a desirable active agent release behavior, but may dissolve or disintegrate too slowly.

Thin films that are microperforated or perforated have been proposed in the past. For example, there are reports for the use of perforated films for dental care, wherein the perforations are intended, e.g., to enable the right position of the films in the oral cavity.

However, up to date, little attention appears to have been paid to improving the haptics of a film delivery system, and in particular by incorporating perforations. Improved haptics can render the application of the films more convenient for patients, and even thick and/or inflexible films could be made acceptable to be administered, allowing higher drug loading as well as a larger selection of materials to be used in such films. A faster dissolution of the films without having to change the polymeric material on the other hand would equally provide the possibility of using materials such as polymers that would otherwise dissolve too slowly to be useful as a matrix for the drug-containing film.

In summary, film delivery systems are promising new drug delivery systems addressing disadvantages of conventional oral dosage forms, which, however, face certain limitations concerning the possible materials to be used, and the size, thickness and drug loading that can be realized when taking into account patient compliance and dissolution behavior.

There is thus a need in the art for improved film delivery system in terms of a controllable dissolution behavior in combination with high drug loading capacity and improved haptics at the same time.

### OBJECTS AND SUMMARY OF THE INVENTION

It is an object of the present invention to provide a film delivery system overcoming the above-mentioned disadvantages.

Thus, it is an object of the present invention to provide a film delivery system providing improved haptics such as decreased foreign-body perception as well as less disturbing, smoother and/or more comfortable feeling at the application site.

It is an object of the present invention to provide a dissolvable and/or disintegratable film delivery system with a controlled dissolution/disintegration behavior.

Thus, it is also an object of the present invention to provide such film delivery systems with improved haptics and controlled dissolution/disintegration behavior at the same time.

It is also an object of the present invention to provide a film delivery system with appropriate haptics and/or dissolution/disintegration kinetics despite a higher thickness and/or higher stiffness of the film.

It is also an object of the present invention to provide a film delivery system for the administration of active ingredients, and in particular for the transmucosal administration of active ingredients with appropriate haptics and/or dissolution/disintegration kinetics despite a higher drug loading which is, e.g., sufficient for achieving an effective dose, and in particular a therapeutically effective dose.

It is another object of the present invention to provide a film delivery system providing appropriate adhesion to the mucosa, e.g. initially but also over time.

These objects and others are accomplished by the present invention, which according to one aspect relates to a film delivery system comprising a mucoadhesive layer, wherein the mucoadhesive layer comprises a film-forming agent and is provided with 5 to 100 perforations per cm².

According to certain embodiments of the invention, the film delivery system according to the invention is for use in a method of treatment.

According to other embodiments, the present invention relates to a method of treatment, wherein the film delivery system according to the invention is administered to a human patient.

According to yet other embodiments, the present invention relates to the use of the inventive film delivery system for the manufacture of a medicament for treating a human patient.

According to yet another aspect, the invention relates to a process of manufacture of a film delivery system comprising a mucoadhesive layer, comprising the steps of:
i) preparing a mucoadhesive layer; and
ii) providing the mucoadhesive layer with 5 to 100 perforations per cm² by using a perforating tool.

According to a certain aspect, the invention also relates to a film delivery system obtainable by such a process of manufacture.

According to another aspect, the invention also relates to a film delivery system comprising a mucoadhesive layer, wherein

According to another aspect, the invention also relates to a film delivery system comprising a mucoadhesive layer, wherein
the mucoadhesive layer comprises a dissolvable film-forming agent and is provided with at least 10 to less than 20 perforations per cm²,
the perforations have a surface area per area of the mucoadhesive layer of at least 10% and 30% or less,
the perforations have a perimeter per area of the mucoadhesive layer of at least 4 cm/cm² and 15 cm/cm² or less,
the perforations all have a circular form and are distributed evenly, and
the mucoadhesive layer has an area weight of at least 200 g/m² and 400 g/m² or less.

According to yet another aspect, the invention also relates to a film delivery system comprising a mucoadhesive layer, wherein
the mucoadhesive layer comprises a dissolvable film-forming agent and is provided with at least 50 to less than 60 perforations per cm²,
the perforations have a surface area per area of the mucoadhesive layer of at least 5% and 30% or less,
the perforations have a perimeter per area of the mucoadhesive layer of at least 8 cm/cm² and 15 cm/cm² or less,
the perforations all have a circular form and are distributed evenly, and
the mucoadhesive layer has an area weight of at least 200 g/m² and 400 g/m² or less.

Within the meaning of this invention, the term “film delivery system”, sometimes also referred to as “film dosage form” or “film therapeutic system”, refers to a system for the administration of an active ingredient consisting of one or more layers. The film delivery system comprises a mucoadhesive layer and is applied to the mucosa such as the oral (e.g., buccal, sublingual, gingival, palatal), vaginal, rectal, nasal or ocular mucosa. It refers to the entire individual dosing unit that is applied to the mucosa of a patient, and in particular comprises an effective amount and in particular a therapeutically effective amount of an active ingredient in the mucoadhesive layer or one of the further layers. The mucoadhesive layer may be located on a release liner (a detachable protective layer), thus, the film delivery system may further comprise a release liner.

Within the meaning of this invention, the term “film delivery system” includes dosage forms intended to provide active delivery also or primarily by the peroral route, in which case the film may serve as a convenient form of oral administration allowing easy swallowing also without the need for water or any beverages to aid in taking the medication so that the films can be easily taken “on the go”. On the other hand, a “film delivery system” in particular refers to a system aiming at transmucosal administration, providing in particular passive transmucosal delivery excluding active transport as in methods including microporation. In case the film is applied in the oral cavity of a patient, enteral delivery by swallowing the active ingredient is also possible and not necessarily undesirable in the film delivery system. With a film delivery system, an active ingredient can be administered not only transmucosally to the systemic circulation, but also only locally limited (to the mucosa at the administration site and/or to tissue which is adjacent to the administration site).

Within the meaning of this invention, the term “transmucosal” refers to the route of active ingredient delivery through the mucosal tissue. Accordingly, the term “transmucosal therapeutic system” or “transmucosal delivery system” refers to a system by which the active ingredient is intended to be administered to the systemic circulation primarily via transmucosal delivery, i.e., through the mucosa, by application to the mucosa, and in particular to the mucosa of the oral cavity. It refers to the entire individual dosing unit that is applied to the mucosa of a patient, and which comprises an effective amount and in particular a therapeutically effective amount of active ingredient in a mucoadhesive layer or one of the further layers, as outlined above for film delivery systems in general.

While transmucosal therapeutic systems can be regarded as a type of “oral thin film”, within the meaning of this invention, “oral thin films” include also film delivery systems for which the focus is less on mucoadhesivity but more on a fast dissolution/disintegration in the saliva (sometimes referred to as “flash wafers”). The intended delivery route for such fast-disintegrating oral thin films or flash wafers may be transmucosal as well as peroral/enteral, and the patient may be instructed, e.g., to place the film sublingually or buccally and not to swallow until dissolved, or to place the film on the tongue and to swallow, in order to favorize the one or the other delivery route.

Within the meaning of this invention, the term “mucoadhesive layer” refers to one of the layers constituting the film delivery system, which comprises a film-forming agent which provides the necessary cohesion and stability to the film, and which has at least one uncovered side which is intended to be applied to the mucosa of the patient. In other words, the mucoadhesive layer in the sense of the present invention is a mucosa-contacting layer. The area of release is provided by the area of the active ingredient-containing layer. The mucoadhesive layer does not need to be at the same time the active ingredient-containing layer, but may be used additionally to enhance adhesion. The sizes of the mucoadhesive layer and the active ingredient-containing layer are usually coextensive (i.e., they are of the same size/dimension and form) and correspond to the area of release. The film delivery system in its most simple form consists of the active ingredient-containing mucoadhesive layer and does not comprise any further layers. The mucoadhesive layer is the final, solidified layer, e.g., obtained after coating and drying a solvent-containing coating composition by a film casting process, or after extruding a film-forming agent or a mixture comprising a film-forming agent.

Terms such as “dissolution”, “dissolvable”, “dissolve” and the like with respect to the film delivery system, any layer of the film delivery system such as the mucoadhesive layer and with respect to the film-forming agent when casted into a film, are to be understood very broadly, and not in the strict scientific sense of chemically dissolving a molecule in a solvent. Any transformation of the solid state of the layer concerned to a liquid state, such as dispersing, forming of a suspension, gelling of the film and disintegrating into smaller parts of gel, etc. has to be regarded as “dissolving” in the sense of the present invention, as long as the “dissolved” material is able to freely move around in the liquid (e.g. saliva). In preferred embodiments, the meaning is limited to the usual chemical sense of dissolving a molecule in a solvent. It should be noted that the term “dissolve” with respect to substances per se, such as any excipients, will continue to be used in the usual chemical sense of dissolving a molecule in a solvent. E.g., the film-forming agent per se can be present in the coating composition during manufacture of the film delivery system in dissolved form in the common chemical sense (e.g. is not dispersed, in form of small parts of gel, etc.), but where the film-forming agent is casted into a film, “dissolving” such a film also includes gelling of the film and disintegrating into smaller parts of gel, i.e., a “dissolvable” film forming layer also has to be understood in a very broad sense.

The mucoadhesive layer may also be manufactured by laminating two or more such solidified layers (e.g., dried layers) of the same composition to provide the desired area weight. The mucoadhesive layer as indicated above is intended to be applied to the mucosa where it should provide adequate adhesion. I.e., within the meaning of this invention, the term “mucoadhesive” refers to a material that in particular adheres to and upon contact with a mucosa, but which preferably is non-tacky and can be touched, e.g., with the fingers and manipulated, e.g., for application into the oral cavity, without unintentionally adhering to the skin of the fingers, when in dry state. A mucoadhesive layer, when in contact with the mucosa, is “self-adhesive”, i.e., provides adhesion to the mucosa so that typically no further aid for fixation is needed. The adhesion strength is preferably strong enough that typical movements in the oral cavity are not sufficient to displace a mucoadhesive layer adhered to the mucosa.

As used herein, the expression “active ingredient” refers to any substance of interest to be delivered by the film delivery system to provide a beneficial or desirable effect on the condition of the subject's body either systemically or locally at the delivery site. An active ingredient in particular includes biologically or pharmacologically active compounds, which may also be referred to as active, drug substance, drug, active ingredient, active pharmaceutical ingredient (API), or the like. Within the meaning of this invention, the term “effective amount” or “therapeutically effective amount” refers to a quantity of active ingredient in the film delivery system sufficient to provide, if administered by the film delivery system to a patient, the desired (therapeutic) effect, e.g., as determined by blood levels of a similar range (e.g. of about 10% to about 1000% as measured as an AUC) when compared to blood levels obtained after a one-time administration of approved drug products of the same active ingredient, or an amount of active similar (e.g. of about 10% to about 1000%) to the amount of active contained in other, topically acting approved drug products of the same active ingredient.

Within the meaning of this invention, the term “administration” refers to the application of the dosage form, i.e., the film delivery system, to the oral (e.g., buccal, sublingual, gingival, palatal), vaginal, rectal, nasal or ocular mucosa, and in particular to the oral mucosa of the patient, which is then maintained on the mucosa for a specified period of time of administration duration, or until the active ingredient-containing layer is dissolved, eroded or substantially disintegrated.

Within the meaning of this invention, the term “area weight” refers to the dry weight of a specific layer, e.g., of the active ingredient-containing layer, provided in g/m². The area weight values are subject to a tolerance of ±10%, preferably ±5% of the nominal value, due to manufacturing variability.

The unit “%” may refer to a percentage given in weight per volume (w/v), volume per volume (v/v) or in weight-%, and, if not indicated otherwise, “%” preferably refers to weight-%.

Within the meaning of this invention, the term “polymer” refers to any substance or material consisting of so-called repeating units obtained by polymerizing one or more monomers, and includes homopolymers which consist of one type of monomer and copolymers which consist of two or more types of monomers. Polymers may be of any architecture such as linear polymers, star polymer, comb polymers, brush polymers, of any monomer arrangements in case of copolymers, e.g. alternating, statistical, block copolymers, or graft polymers. The minimum molecular weight varies depending on the polymer type and is known to the skilled person. Polymers may e.g. have a molecular weight above 2,000, preferably above 5,000 and more preferably above 10,000 Dalton. Correspondingly, compounds with a molecular weight below 2,000, preferably below 5,000 or more preferably below 10,000 Dalton are usually referred to as oligomers.

Within the meaning of this invention, the term “room temperature” refers to the unmodified temperature found indoors in the laboratory where the experiments are conducted and usually lies within 15 to 35° C., preferably about 18 to 25° C.

Within the meaning of this invention, the term “patient” refers to a subject who has presented a clinical manifestation of a particular symptom or symptoms suggesting the need for treatment, who is treated preventatively or prophylactically for a condition, or who has been diagnosed with a condition to be treated.

Within the meaning of this invention, the term “coating composition” refers to a composition comprising all components of one of the layers such as the mucoadhesive layer in a solvent, which may be coated to form the corresponding layer such as the mucoadhesive layer upon drying.

Within the meaning of this invention, the term “solvent” refers to any liquid substance, which preferably is a volatile organic liquid such as methanol, ethanol, isopropanol, acetone, ethyl acetate, methylene chloride, hexane, n-heptane, heptanes, toluene and mixtures thereof.

Within the meaning of this invention, and unless otherwise specified, the term “about” refers to an amount that is ±10% of the disclosed amount. In some embodiments, the term “about” refers to an amount that is ±5% of the disclosed amount. In some embodiments, the term “about” refers to an amount that is ±2% of the disclosed amount.

Within the meaning of this invention, the term “substantial” or “substantially” is used to refer to a large part of the corresponding object or component concerned, e.g., a composition “consisting substantially of” a component comprises a large amount of such a component, such as at least 95% by weight, preferably at least 98% or even at least 99% by weight.

Within the meaning of this invention, the term “surface area” refers to the maximum area of a perforation when regarded from a top view, i.e., in case of a conical perforation, the surface area is to be calculated based on the shape at the surface of the piercing side.

Within the meaning of this invention, a “triangular”, “rectangular”, “staggered” or “diagonal” pitch refers to the shape formed when regarding the pitch of closest neighbouring perforations in an arrangement wherein the perforations are distributed evenly (see FIG. 10a to 10d).

Within the meaning of this invention, the “margin” refers to an unperforated circumferential edge of the mucoadhesive layer.

### BRIEF DESCRIPTION OF THE DRAWINGS

FIG. 1 is a schematical drawing of a film delivery system of the current invention from a top view with a mucoadhesive layer provided with perforations in circular form in an 8*8 checkerboard grid pattern (i.e., arranged in a rectangular pitch) provided with a margin (pattern A).

FIG. 2 depicts a film delivery system consisting of a mucoadhesive layer perforated according to the perforation pattern A.

FIG. 3 is a schematical drawing of a film delivery system of the current invention from a top view with a mucoadhesive layer provided with perforations in circular form in an 10*10 checkerboard grid pattern (i.e., arranged in a rectangular pitch) provided with a margin (pattern B).

FIG. 4 depicts a film delivery system consisting of a mucoadhesive layer perforated according to the perforation pattern B.

FIG. 5 is a schematical drawing of a film delivery system of the current invention from a top view with a mucoadhesive layer provided with perforations in circular form in an 19*19 checkerboard grid pattern (i.e., arranged in a rectangular pitch) provided with a margin (pattern B).

FIG. 6 depicts a film delivery system consisting of a mucoadhesive layer perforated according to the perforation pattern C.

FIG. 7a depicts in a bird's eye view a single perforation in the sense of the present invention which is a through-hole in circular form and cylindrical.

FIG. 7b depicts in a bird's eye view a single perforation in the sense of the present invention which is a full cylindrical slit.

FIG. 7c depicts from a side view a single perforation in the sense of the present invention which is a full cylindrical slit or a cylindrical through-hole.

FIG. 7d depicts from a side view a single perforation in the sense of the present invention which is a full cylindrical slit or a cylindrical through-hole, which shows a slightly widened opening at the piercing side and a frayed edge at the opposite side with fringes formed from the perforating tool pushing out material when piercing through the layer.

FIG. 8a depicts in a bird's eye view a single perforation in the sense of the present invention which is a through-hole in circular form and conical.

FIG. 8b depicts from a top view a single perforation in the sense of the present invention which is through-hole in circular form and conical, showing also the larger circular shape forming the basis for calculating the surface area.

FIG. 8c depicts from a side view a single perforation in the sense of the present invention which is a full conical slit or a conical through-hole.

FIG. 9a depicts in a bird's eye view a single perforation in the sense of the present invention which is a cylindrical pocket-hole in circular form.

FIG. 9b depicts from a side view a single perforation in the sense of the present invention which is a cylindrical pocket-hole in circular form.

FIG. 9c depicts in a bird's eye view a single perforation in the sense of the present invention which is a conical incomplete slit.

FIG. 9d depicts from a side view a single perforation in the sense of the present invention which is a conical incomplete slit.

FIG. 10a depicts from a top view an arrangement of perforations which are distributed evenly in a triangular pitch.

FIG. 10b depicts from a top view an arrangement of perforations which are distributed evenly in a rectangular pitch.

FIG. 10c depicts from a top view an arrangement of perforations which are distributed evenly in a staggered pitch.

FIG. 10d depicts from a top view an arrangement of perforations which are distributed evenly in a diagonal pitch.

The Figures are for illustrative purposes only and are neither intended to limit the claimed scope nor are they true to scale.

### DETAILED DESCRIPTION

### Film Delivery System

The present invention is related to a film delivery system comprising a mucoadhesive layer.

The film delivery system is intended to be used for the administration of an active ingredient useful in the treatment and/or prophylaxis of diseases and medical conditions and is applied to the mucosa of a patient. The mucoadhesive layer comprises a film-forming agent and is provided with 5 to 100 perforations per cm².

Thus, in accordance with the present invention, the film delivery system comprises a mucoadhesive layer that comprises a film-forming agent and is provided with 5 to 100 perforations per cm².

The inventive film delivery system consists of one or more thin layers and the active ingredient may be contained in the mucoadhesive layer and/or in one or more of the further layers, which would then form a separate active ingredient-containing layer. In other words, the mucoadhesive layer does not need to be at the same time the active ingredient-containing layer, but may be used additionally to enhance adhesion. On the other hand, the film delivery system is simpler and easier in manufacture if the mucoadhesive layer is the only active-ingredient containing layer, and in its most simple form the film delivery system consists of an active ingredient-containing mucoadhesive layer and does not comprise any further layers. In certain embodiments, the film delivery system may comprise further layers such as a backing layer which prevents the active ingredient from being released from the therapeutic system at its non-mucosally adhering surface into the saliva, and thus is considered to be able to prevent or reduce unintended delivery via the gastrointestinal route, an adhesive layer that might serve to keep layers such as the backing layer and the active ingredient-containing layer adhered to each other, or a cosmetic layer which may provide for a decorative means such as coloring or imprinting and/or prevent the patient from touching the other layers.

Thus, in some embodiments, the film delivery system comprises or does not comprise a cosmetic layer. In some embodiments, the film delivery system comprises or does not comprise a backing layer. It is understood that such additional layers may also have perforations, which will be advantageously formed at the same time as those of the mucoadhesive layer, and thus will be part of the same perforation extending through two or more layers, and preferably all layers of the film delivery system. In addition, in case the film delivery system comprises such additional layers, and in particular in the case a backing layer is present, the film delivery system may be provided with a means to indicate which side has to be applied to the mucosa. Such means could be a one-sided coloring or imprinting, or the presence of a detachable release liner. As will be further outlined below, also in case the film delivery system has no additional layers and consists of the mucoadhesive layer, e.g., in case the perforation does not extend through the whole layer thickness, or one of the sides shows frayed edges of the perforations, one of the two sides may be preferred over the other for being applied to the mucosa. In such a situation, the film delivery system can be provided with a means to indicate which side has to be applied to the mucosa, which may be those as referred to above.

In yet other embodiments, the film delivery system is a mucoadhesive delivery system.

In view of convenience of manufacture and restricting the overall system size in terms of surface area, preferably all layers of the film delivery system are coextensive, i.e., are identical in shape and size, so that the area of release in such a case corresponds to the area defined by the film layers. The film preferably is large enough to allow a patient convenient manipulating with the fingers without the aid of any specific device such as a tweezer, and a certain minimum size is also required in order to ensure that the film does not detach prematurely from the mucosa, and also for being able to include a sufficient amount of active ingredient without having to use very thick films. On the other hand, if the film is too large, it will be uncomfortable to apply and to wear, leading to low patient compliance. Considering this, in certain embodiments of the invention, the film delivery system has an area of release of at least 0.2 cm², preferably at least 0.5 cm², 1 or 2 cm², or has an area of release of less than or equal to 10 cm², preferably less than or equal to 7 cm², 6 or 5 cm², or has an area of release of from 0.2 to 10 cm², and more preferably of from 0.5 to 7 cm², 1 to 6 cm² or 2 to 5 cm². In certain further embodiments, the film delivery system has a total weight of at least 5 mg or 10 mg, of less than or equal to 175 mg or 150 mg, or has a total weight of from 5 to 175 mg or from 10 to 150 mg.

As outlined also above, the inventive film delivery system consists of one or more thin layers, and is in the form of a film which may have a circular, rectangular or square shape.

The film preferably has a certain degree of thickness, as otherwise it will be difficult to incorporate the required amount of active ingredient, and as very thin films are not easy to manufacture, in particular with respect to providing an even thickness. Without wishing to be bound by theory and as will be further elaborated further below in the chapter concerning the mucoadhesive layer, the presence of the perforations in the mucoadhesive layer results in improved haptics of the film delivery system as well as a possible acceleration of the dissolution/disintegration of the film, which means that the film delivery system may be thicker without causing discomfort to the patient when compared to conventional film delivery systems without perforations. Thus, in certain embodiments, the film delivery system is in the form of a thin film having a total area weight (disregarding any release liner) of at least 80 g/m², preferably at least 120 g/m², or more preferably at least 250 g/m². Regarding its thickness, the mucoadhesive layer structure is in the form of a thin film having a total thickness of at least 50 μm, preferably at least 100 μm, and more preferably at least 200 μm. On the other hand, very thick films will be still perceived by the patient as disturbing objects in the oral cavity, and thus are disadvantageous in terms of patient compliance. Thus, in certain embodiments, the film delivery system is in the form of a thin film having a total area weight of less than or equal to 900 g/m², preferably less than or equal to 700 g/m², or more preferably less than or equal to 500 g/m² or most preferably less than or equal to 300 g/m². Or, in terms of thickness, the mucoadhesive layer structure is in the form of a thin film having a total thickness of less than 700 μm, preferably less than 500 μm, and more preferably less than 400 μm. In preferred embodiments, the film delivery system is in the form of a thin film having a total area weight of from 80 to 900 g/m², preferably from 120 to 700 g/m², or more preferably from 250 to 500 g/m² or most preferably from 250 to 300 g/m², or having a total thickness from 50 to 700 μm, preferably from 100 to 500 μm, and more preferably from 200 to 400 μm.

The film delivery system according to the invention can be stored in any conventional packaging known to the skilled person, such as a seam-sealed pouch without any further means of protection. Also, the film delivery system may or may not be located on a detachable protective layer (release liner) from which it is removed immediately before application to the mucosa of the patient's oral cavity. The packaging may be child resistant and/or senior friendly.

### Mucoadhesive Layer

As outlined in more detail above, the film delivery system according to the present invention comprises a mucoadhesive layer that comprises a film-forming agent. As also explained in the above sections, film delivery systems of the past have been limited in its application mainly due to the limitation in thickness and film-forming material imposed by the dissolution behavior as well as patient compliance in terms of possible discomfort due to thick and/or inflexible films required to make such a film delivery system viable in practice. The film delivery system of the present invention addresses these concerns by improving haptics and/or dissolution/disintegration behavior of the film by providing the mucoadhesive layer with a 5 to 100 perforations per cm².

Thus, the mucoadhesive layer comprises:
i) a film-forming agent; and
ii) 5 to 100 perforations per cm².

In analogy to what has been indicated for the whole film delivery system, the mucoadhesive layer preferably is an active-ingredient containing layer. In such a case, the mucoadhesive layer preferably has a certain degree of thickness allowing to incorporate the required amount of active ingredient, and avoiding very thin films which are not easy to manufacture, in particular with respect to providing an even thickness. Since the haptics is improved and the dissolution/disintegration of the film is possibly accelerated by way of the perforations, the mucoadhesive layer may be thicker without causing discomfort to the patient when compared to conventional film delivery systems without perforations. Thus, in certain embodiments, the mucoadhesive layer has an area weight of at least 70 g/m², at least 100 g/m², or at least 200 g/m². In certain embodiments, the mucoadhesive layer has an area weight of 700 g/m² or less, 500 g/m² or less, 400 g/m² or less or 300 g/m² or less. In certain embodiments, the mucoadhesive layer has an area weight of from 70 g/m² to 700 g/m², from 100 g/m² to 500 g/m², from 200 g/m² to 400 g/m² or from 200 g/m² to 300 g/m².

The process of manufacture of the film delivery system will be explained in full detail further below but comprises preparing the mucoadhesive layer, e.g., by a hot-melt process or by a film casting process. Thus, in certain embodiments, the mucoadhesive layer is obtainable by a hot-melt process, whereas in other certain embodiments of the invention, the mucoadhesive layer is obtainable by a film casting process.

### Perforations

In accordance with the present invention, the mucoadhesive layer comprised in the film delivery system is provided with 5 to 100 perforations per cm². It has been quite surprisingly shown by way of experiments of which the details can be found further below, that film delivery systems with a mucoadhesive layer provided with such perforations are improved in terms of haptics, i.e., such film delivery systems are more comfortable to wear for the patient, but also in terms of dissolution/disintegration kinetics in that they dissolve/disintegrate faster when compared to film delivery systems having no perforations. While such advantageous properties were unexpected, without wishing to be bound by theory, it is believed that the perforations not only provide an enlarged surface area but also result in the mucoadhesive layer consisting rather of a network of remaining “bridges”. This way, a faster dissolution/disintegration is achieved when getting into contact with the wet mucosa, which in turn results in a faster softening of the dry and thus to a certain extent stiff film, and the softened film is more comfortable for the patient, in particular when taking into account that only the remaining “bridges” of the network need to be softened.

As indicated above, the mucoadhesive layer is provided with 5 to 100 perforations per cm². In particular embodiments, the mucoadhesive layer is provided with at least 7, at least 10, at least 20 or at least 50 perforations per cm² or with less than 90, less than 80 or less than 60 perforations per cm² or the mucoadhesive layer is provided with 7 to less than 90, 10 to less than 80, 20 to less than 60 or 50 to less than 60 perforations per cm².

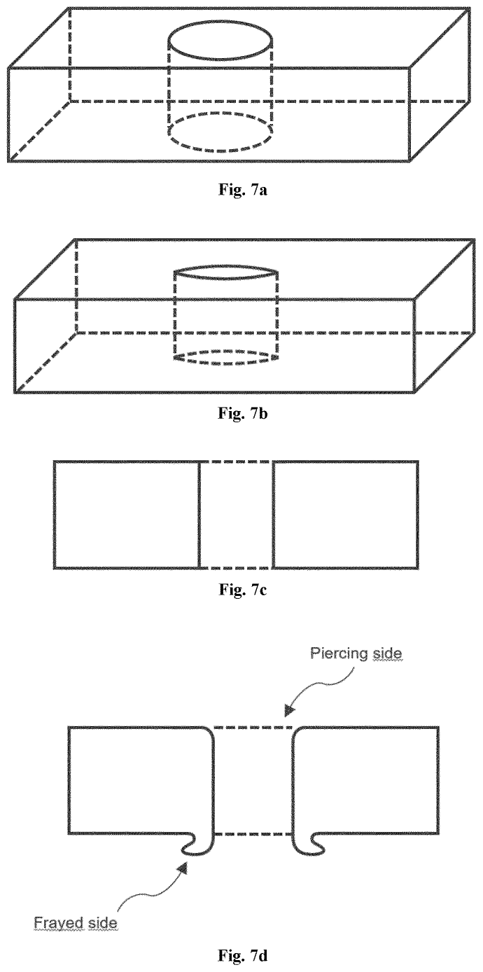

While the perforations can have any dimensions and form both in terms of the shape of the perforation at the surface of the mucoadhesive layer as well as the cross-sectional shape through the mucoadhesive layer, certain forms are preferable in particular in view of the ease of manufacture. I.e., holes can be easily prepared by employing a perforating tool selected from needle or punching devices, while slits can be prepared easily by using a cutting device as perforating tool. Exemplary forms and shapes of perforations in accordance with the present invention are shown in FIG. 7a to 7d, 8a to 8c as well as 9a to 9d and will be explained in the following.

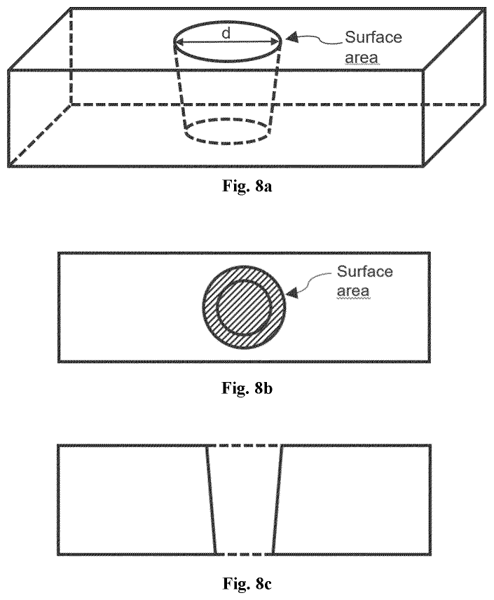

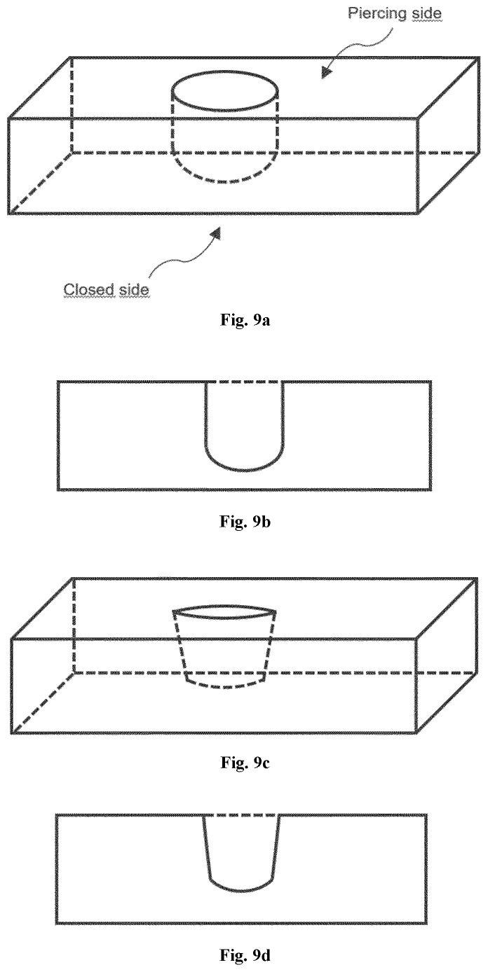

Depending on the depth the mucoadhesive layer is pierced with the perforating tool, the perforation can span the whole thickness of the mucoadhesive layer which is shown in FIGS. 7a to 7d as well as 8a to 8c (a through-hole or a full slit) so that there is no residual thickness or, as shown in FIG. 9a to 9c, only a part thereof (pocket-hole in FIG. 9a and FIG. 9b or incomplete slit in FIG. 9c and FIG. 9d) so that the perforation has residual thickness. In particular, the cutting device may cut a perforation in the form of a full slit by full-depth cutting as shown in FIG. 7b, or a perforation in the form of an incomplete slit by controlled-depth cutting as shown in FIG. 9c and FIG. 9d, the needle device may incorporate a perforation in the form of a through-hole by piercing through the mucoadhesive layer as shown in FIG. 7a and 8a to 8c, or a perforation in the form of a pocket-hole by controlled-depth piercing the mucoadhesive layer as shown in FIGS. 9a and 9b, while a punching device may punch a perforation and thereby removes the punched part from the mucoadhesive layer.

Thus, in certain embodiments, the perforations are in the form of a hole, and in particular a through-hole or a pocket-hole, or in the form of a slit, and in particular a full slit or an incomplete slit and/or wherein the perforations have a circular, an oval, a triangular, a rectangular or a hexagonal form. The perforations may all have the same form, or two or more different forms. The perforations may also have a circular, an oval, a triangular, a rectangular or a hexagonal form to a substantial part, e.g., more than 50%, 70%, 90% or all perforations may have a form selected from a circular, an oval, a triangular, a rectangular and a hexagonal form, and in particular may have a circular form. The shape of the perforation from top view may be identical throughout the layer thickness so that the perforation is cylindrical (FIGS. 7a, 7b, 9a and 9b), or the perforation tapers and is conical (FIGS. 8a, 8b, 9c and 9d), or may have any other change of shape within the layer thickness. In case the perforation spans only a part of the layer thickness, one side is closed and the perforation opening is at the piercing side. In certain embodiments, the film delivery system is intended to be applied with the piercing side as the mucosa-contacting side, while the closed side is intended to face away from the mucosa. In certain embodiments, the perforation has a frayed edge on one side. Such a frayed edge can be the result of the perforation being introduced by piercing with a needle device whereby the material of the pierced layer is pushed out at the opposite side and forms fringes resulting in such a frayed edge (FIG. 7d). The frayed edge might protrude from the surface of the mucoadhesive layer and thus, while both sides of the mucoadhesive layer can be applied to the mucosa, in view of an improved haptics, it is the piercing side and not the frayed side that is preferably applied to the mucosa. In such situations, the film delivery system can be provided with a means to indicate which side has to be applied to the mucosa, which may be those as referred to above.

In terms of dimension, the perforations are limited only in that the number of perforations should be within 5 to 100 per cm², and that the film delivery system has a practical limitation in size. Thus, in certain embodiments of the invention, the perforations have a surface area of at least 0.0005 cm², at least 0.001 cm² or at least 0.002 cm² per perforation or the perforations have a surface area of 0.15 cm² or less, 0.05 cm² or less or 0.03 cm² or less per perforation. In certain embodiments, the perforations have a surface area per area of the mucoadhesive layer of at least 2%, at least 5% or at least 10% or the surface area per area of the mucoadhesive layer is 70% or less, 50% or less or 30% or less. The surface area per area of the mucoadhesive layer can be calculated, for example for a film delivery system wherein each perforation has one and the same form, by multiplying the surface area for one perforation by the total number of perforations, and then dividing the resulting value by the total area of the mucoadhesive layer. If the surface area per area of the mucoadhesive layer is too low, the advantageous effects of improved haptics and dissolution/disintegration properties might be less pronounced. On the other hand, if the surface area per area of the mucoadhesive layer is too high, the integrity and stability of the film might be affected, and also the amount of film material might be decreased in such an extent that it is not possible to incorporate a sufficient amount of active ingredient.

In certain embodiments of the invention, the perforations have a perimeter of at least 0.1 cm, at least 0.2 cm or at least 0.3 cm per perforation or the perforations have a perimeter of 2 cm or less, 1 cm or less, or 0.8 cm or less per perforation. In certain embodiments, the perimeter per area of the mucoadhesive layer is at least 2 cm/cm², at least 4 cm/cm² or at least 8 cm/cm² or the perimeter per area of the mucoadhesive layer is 30 cm/cm² or less, 20 cm/cm² or less or 15 cm/cm² or less. The perimeter per area of the mucoadhesive layer can be calculated, for example for a film delivery system wherein each perforation has one and the same form, by multiplying the perimeter for one perforation by the total number of perforations, and then dividing the resulting value by the total area of the mucoadhesive layer. A longer perimeter is believed to be beneficial for accelerating the dissolution behavior by increasing the sidewall area of the hole or slit, but on the other hand if the perimeter gets too long, again the stability of the film might be negatively affected.

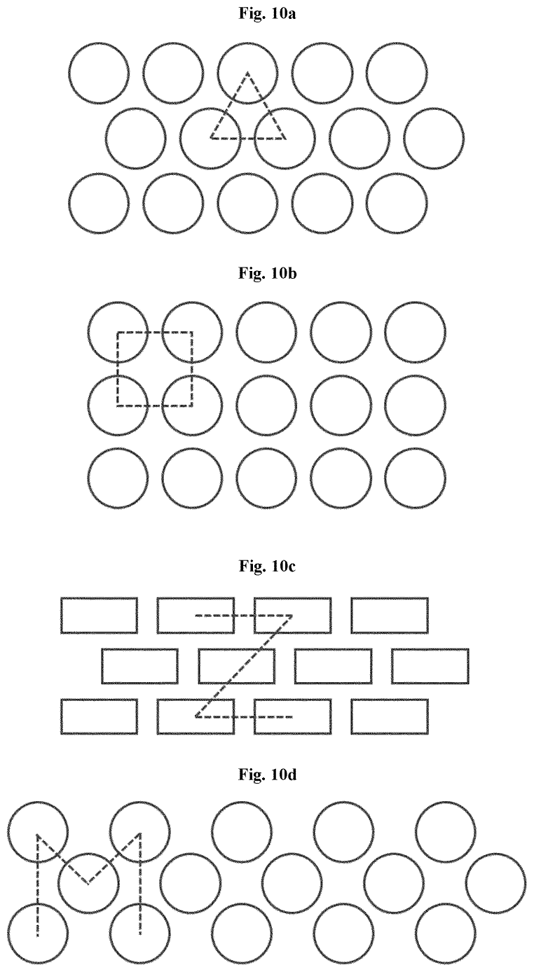

In certain embodiments of the invention, the perforations are distributed evenly or non-evenly. If the perforations are distributed evenly, the perforations may be arranged in a triangular, a rectangular, a staggered or a diagonal pitch. Such an even distribution is exemplarily depicted in FIG. 10a (triangular pitch), in FIG. 10b (rectangular pitch), in FIG. 10c (a staggered) or in FIG. 10d (diagonal pitch).

In certain embodiments of the invention, the mucoadhesive layer has a margin, which is free of perforations, or has no margin. A margin might have the advantage of providing a more stable edge that can be manipulated with the fingers, e.g., for unpeeling from a packaging pouch, without the risk of tearing or breaking the film.

As outlined above, the perforations may be prepared by employing an appropriate perforating tool. Thus, in certain embodiments of the invention, the perforations are incorporated by using a perforating tool, and such perforating tool may be selected from cutting devices, needle devices and punching devices.

Needle devices in the sense of the present invention may be selected from the group consisting of needles, needle assemblies, and pin rollers. They may also be provided with needles or pins that have a diameter of at least 0.1, 0.2, 0.3 or 0.5 mm or a diameter of less than 3 mm or less, 2 mm or less, or 1 mm or less. Moreover, the needle device may have a blunt tip or a sharp needle head. The needle device incorporates a perforation in the form of a through-hole by piercing through the mucoadhesive layer, or a perforation in the form of a pocket-hole by controlled-depth piercing the mucoadhesive layer.

### Active Ingredient

As outlined above, the film delivery system according to the present invention comprises an active ingredient, and the active ingredient may be contained in the mucoadhesive layer and/or in one or more of the further layers.

The active ingredient can be any substance of interest to be delivered by the film delivery system to provide a beneficial or desirable effect on the condition of the subject's body either systemically or locally at the delivery site. The active ingredient is in particular selected from any known active pharmaceutical ingredient, supplements such as food, dietary and/or nutritional supplements including vitamins, minerals, enzymes, probiotics, etc., substances for oral care and/or oral hygiene such as substances acting against bad breath, as well as lifestyle medicaments such as stimulants, natural extracts and substances for a better sleep or against snoring.

### Film-Forming Agent

As outlined above, the film delivery system according to the present invention comprises a mucoadhesive layer comprising a film-forming agent.

The film-forming polymers that can be used in the present invention are not particularly limited and any known polymer with film-forming properties, preferably those that are pharmaceutically acceptable (and, e.g., are approved for pharmaceutical applications), can be employed. The film-forming agent may in particular be a dissolvable film-forming agent.

This film-forming agent forms a matrix and provides for sufficient cohesion of the mucoadhesive layer as long as it is kept in dry state. According to certain embodiments, the film-forming agent may also provide for sufficient adhesion to the mucosa once wet, i.e. when having been brought in contact with the mucosa. In such embodiments, but also in general, the film-forming agent may be selected from mucoadhesive polymers.

The film-forming agent is the primary control over the dissolution/disintegration behavior of the mucoadhesive layer. By choosing an appropriate film-forming agent, the adhesion to the mucosa as well as disintegration behavior can be appropriately adjusted, e.g. in terms of the disintegration time but also in terms of integrity of the film delivery system. In such embodiments, but also in general, the film-forming agent may be selected from dissolvable mucoadhesive polymers.

In certain embodiments, the film-forming agent can be a polymer, a natural film-forming agent, or any mixture thereof.

Film-forming agents which are suitable in accordance with the invention are e.g. selected from the group consisting of polymers such as polyvinylpyrrolidone (commercially available as Kollidon® 30F from BASF), methyl cellulose (commercially available as Methocel® from Colorcon), ethyl cellulose (commercially available as Ethocel® from Colorcon), hydroxyethyl cellulose (commercially available as Natrosol® 250 L from Ashland Industries), hydroxypropyl cellulose (commercially available as Klucel® from Ashland Industries), hydroxypropylmethyl cellulose (also known as hypromellose, commercially available as Pharmacoat® from Shin-Etsu), carboxymethyl cellulose sodium (uncrosslinked sodium salt of carboxymethyl cellulose also referred to as CMC or carmellose, commercially available as Blanose® from Ashland Industries), polyethylene glycol-polyvinyl acetate- and polyvinylcaprolactame-based graft copolymers (commercially available as Soluplus® from BASF), polyvinyl alcohol (commercially available as Mowiol® from Kuraray or, e.g. as Parteck® MXP, from Merck), polyvinyl alcohol-polyethylene glycol copolymers (commercially available as Kollicoat® IR from BASF), polyvinylpyrrolidone-polyvinylacetate copolymers (also referred to as copovidones and commercially available e.g. as Kollidon® VA64 from BASF), polyethylene oxides, polyethylene glycols, methacrylic acid-methyl methacrylate copolymers (commercially available as Eudragit® L100, Eudragit® L12,5, Eudragit® S100 and Eudragit® S12,5 from Evonik), and methacrylic acid-ethyl methacrylate copolymers (commercially available as Eudragit® L100-55 and Eudragit® L30D55 from Evonik), and natural film-forming agents such as shellac, pectin, gelatin, alginate, pullulan and starch derivatives, and any mixtures thereof.

In certain embodiments, the film-forming agent is selected from the group consisting of hydroxypropyl cellulose, hydroxypropylmethyl cellulose, polyethylene oxide, polyvinyl alcohol, and any mixture thereof.

In certain embodiments of the invention, the film-forming agent is a hydroxypropyl cellulose.

Hydroxypropyl cellulose is commercially available from Ashland under the brand name Klucel™ and is provided in several grades.

The grades differ from each other by molecular weight MW (as measured by GPC-size exclusion chromatography) and Brookfield viscosity (25° C., LVF, Moisture Free), and are as follows:
The HF grade has a MW of 1,150,000 and a Brookfield Viscosity of 1500-3000 (1% in water),
the MF grade has a MW of 850,000 and a Brookfield Viscosity of 4000-6500 (2% in water),
the GF grade has a MW of 370,000 and a Brookfield Viscosity of 150-400 (2% in water),
the JF grade has a MW of 140,000 and a Brookfield Viscosity of 150-400 (5% in water),
the LF grade has a MW of 95,000 and a Brookfield Viscosity of 75-150 (5% in water),
the EF grade has a MW of 80,000 and a Brookfield Viscosity of 300-600 (10% in water),
the ELF grade has a MW of 40,000 and a Brookfield Viscosity of 150-300 (10% in water).

In certain embodiments, the film-forming agent is a hydroxypropyl cellulose having a molecular weight (as measured by GPC-size exclusion chromatography) of from 30,000 to 1,500,000, from 30,000 to 500,000, or any mixture thereof, and in particular, the hydroxypropyl cellulose has a molecular weight (as measured by GPC-size exclusion chromatography) selected from
between 35,000 and 45,000, in particular 40,000
between 75,000 and 85,000, in particular 80,000
between 90,000 and 100,000, in particular 95,000
between 130,000 and 150,000, in particular 140,000
between 350,000 and 400,000, in particular 370,000,
between 800,000 and 900,000, in particular 850,000,
between 1,100,000 and 1,200,000, in particular 1,150,000,
or any mixture thereof.

In certain embodiments, the film-forming agent is a hydroxypropyl cellulose having a molecular weight of 80,000, 95,000 or 370,000, or any mixture thereof.

In certain embodiments of the invention, the film-forming agent is a hydroxypropylmethyl cellulose.

Hydroxypropyl methylcellulose is commercially available from Dupont under the brand name AFFINISOL™ HPMC HME and is provided in several grades.

The grades differ from each other by molecular weight MW, and are as follows:
the 15 LV grade has a MW of 85,000,
the 100 LV grade has a MW of 180,000, and
the 4M grade has a MW of 550,000.

Thus, in certain embodiments, the film-forming agent is a hydroxypropyl methylcellulose having a MW of 85,000, 190,000, or 550,000, or any mixture thereof.

In certain embodiments of the invention, the film-forming agent is a polyethylene oxide.

Polyethylene oxide is commercially available from Dupont under the brand name POLYOX™ and is provided in several grades.

The grades differ from each other by molecular weight MW, and are as follows:
the N-10 grade has a MW of 100,000,
the N-80 grade has a MW of 200,000,
the N-750 grade has a MW of 300,000,
the 205 grade has a MW of 600,000,
the 1105 grade has a MW of 900,000,
the N-12K grade has a MW of 1,000,000,
the N-60K grade has a MW of 2,000,000,
the 301 grade has a MW of 4,000,000,
the Coagulant grade has a MW of 5,000,000,
the 303 grade has a MW of 7,000,000.

Thus, in certain embodiments, the film-forming agent is a polyethylene oxide having a molecular weight in the range of from 100,000 to 7,000,000, or any mixture thereof, and in particular a polyethylene oxide having a molecular weight selected from the group consisting of 100,000, 200,000, 300,000, 600,000, 900,000, 1,000,000, 2,000,000, 4,000,000, 5,000,000 and 7,000,000, or any mixture thereof.

More preferably, the film-forming agent is a polyethylene oxide having a molecular weight of 100,000 or 200,000, or a mixture thereof.

In certain embodiments of the invention, the film-forming agent is a polyvinyl alcohol.

Polyvinyl alcohol is commercially available from Kuraray under the brand name Mowiol and from Merck, e.g., under the brand name Parteck® MXP, and is provided in different grades. In certain embodiments, the film-forming agent is a polyvinyl alcohol having a molecular weight in the range of from 20,000 to 150,000, or any mixture thereof.

The Mowiol partially hydrolyzed grades differ from each other by molecular weight MW; and are as follows:
the PVA 3-83 has a MW of 14,000
the PVA 4-88 has a MW of 31,000
the PVA 5-88 has a MW of 37,000
the PVA 3-82 has a MW of 47,000
the PVA 8-88 has a MW of 67,000
the PVA 18-88 has a MW of 130,000
the PVA 23-88 has a MW of 150,000
the PVA 26-88 has a MW of 160,000
the PVA 40-88 has a MW of 205,000.
   Whereas the Mowiol fully hydrolyzed grades differ from each other by molecular weight MW and are as follows:
the PVA 3-98 has a MW of 16,000
the PVA 4-98 has a MW of 27,000
the PVA 6-98 has a MW of 47,000
the PVA 10-98 has a MW of 67,000
the PVA 20-98 has a MW of 125,000
the PVA 56-98 has a MW of 195,000
the PVA 28-99 has a MW of 145,000
   The Parteck® MXP 4-88 grade PVA has a MW of 32,000, and the Parteck® MXP 3-82 grade PVA has a MW of 47,000. In these grade names, the first number provides the apparent viscosity in mPa·s of a 4% aqueous solution at 20° C. and the second number provides the hydrolysis grade in %. So, Parteck® MXP 3-82 shows a viscosity of 3 mPa·s and a hydrolysis grade of 82%, while Parteck® MXP 4-88 shows a viscosity of 4 mPa·s and a hydrolysis grade of 88%. Merck similarly offers polyvinyl alcohol grades 5-88, 8-88, 18-88, 26-88, 28-99, and 40-88.

In certain embodiments, the film-forming agent is a polyvinyl alcohol having a molecular weight of 32,000.

In certain embodiments, the amount of the film-forming agent is
at least 40 wt-%, at least 70 wt-%, or at least 80 wt-%,
100 wt-% or less, or 95 wt-% or less, or is
100 wt-% or 90 wt-%
   of the mucoadhesive layer.

### Further Excipients

The layers of the film delivery system according to the invention and in particular the mucoadhesive layer may each comprise further excipients. Such excipients that can be used in the present invention are not particularly limited and any known excipient for pharmaceutical formulations, and in particular those that are pharmaceutically acceptable (and, e.g., are approved for pharmaceutical applications), can be employed.

Thus, in certain embodiments, any one of the layers of the film delivery system and in particular the mucoadhesive layer further comprises one or more excipients selected from the group consisting of fatty acids, sweeteners, flavoring agents, colorants, permeation enhancers, solubilizers, plasticizers, humectants, disintegrants, wetting enhancers, dissolution enhancers, emulsifiers, antioxidants, stabilizers, buffer reagents and further film-forming agents.

In certain embodiments, the plasticizer is selected from the group consisting of mono-, di-, oligo- and polysaccharides and derivatives such as sorbitol, polyethylene glycol, triacetin, triethyl citrate, propylene glycol, glycerol and medium chain triglycerides.

In certain embodiments, the fatty acid is selected from the group consisting of a saturated or unsaturated, linear or branched carboxylic acid comprising 4 to 24 carbon atoms, and in particular is selected from the group consisting of caprylic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid.

In certain embodiments, the disintegrant is selected from cross-linked polyvinyl pyrrolidones.

In certain embodiments, any one of the layers of the film delivery system and in particular the mucoadhesive layer further comprises one or more natural or artificial sweeteners selected from the group consisting of saccharose, glucose, fructose, sorbitol, mannitol, isomalt, maltitol, lactitol, xylitol, erythritol, sucralose, acesulfame potassium, aspartame, cyclamate, neohesperidine, neotame, steviol glycosides, thaumatin and saccharin sodium.

In certain embodiments, any one of the layers of the film delivery system and in particular the mucoadhesive layer further comprises one or more natural or artificial flavoring agents selected from the group consisting of vanillin, methyl salicylate, menthol, manzanate, diacetyl, acetylpropionyl, acetoin, isoamyl acetate, benzaldehyde, cinnamaldehyde, ethyl propionate, methyl anthranilate, limonene, ethyl decadienoate, allyl hexanoate, ethyl maltol, 2,4-dithiapentane, ethylvanillin and eucalyptol as well as flavoring compositions such as peppermint flavor.

### Method of Treatment/Medical Use/Application

In accordance with a specific aspect of the present invention, the film delivery system according to the invention is for use in a method of treatment and/or prophylaxis of diseases and medical conditions, preferably in a human patient. In accordance with another aspect, the present invention is related to a method of treatment, wherein the film delivery system according to the invention is administered to a human patient. In yet another aspect, the present invention relates to the use of the inventive film delivery system for the manufacture of a medicament for a treatment, preferably for the treatment of a human patient.

In certain embodiments, the film delivery system is for application to the mucosa, and in particular to the mucosa of the oral cavity of a human patient. In other words, the film delivery system is administered by applying the film to the mucosa and in particular to the mucosa of the oral cavity of a human patient and maintained on the mucosa for a certain period of time (the administration period), after which the film delivery system is dissolved, eroded or substantially disintegrated, or is removed from the application site. In certain embodiments, the invention is also related to a method of administering the film delivery system according to the invention to the mucosa, and in particular to the mucosa of the oral cavity of a human patient. In certain embodiments, the invention relates to the use of the inventive film delivery system for the manufacture of a medicament for application to the mucosa, and in particular to the mucosa of the oral cavity of a human patient.

In the above embodiments, the film delivery system may be applied to the buccal, sublingual, gingival or palatal mucosa, preferably the buccal mucosa of the oral cavity of a human patient. The administration period may be as short as less than a minute, or several minutes, such as from 1 to 10 minutes, 2 to 10 minutes, a little longer, such as 10 to 30 minutes, or even extended such as from 30 minutes to 60 minutes and even longer. Where the film delivery system is left in the oral cavity and not removed, the administration period is the time until the film delivery system is dissolved, eroded or substantially disintegrated. The dissolution behavior of the film delivery system can be determined in vitro, e.g., by dissolution experiments. Thus, in certain embodiments, at least 5 wt-%, 25 wt-% or 40 wt-%, or less than 90 wt-%, 70 wt-% or 60 wt-%, or from 5 to 90 wt-%, from 25 to 70 wt-%, or from 40 to 60 wt-% of the film delivery system are dissolved after 5 minutes as measured using a USP apparatus 1 (basket) at 37±0.5° C. in 900 mL 0.01 N HCl. In certain embodiments, at least 5 wt-%, 25 wt-% or 40 wt-%, or less than 90 wt-%, 70 wt-% or 60 wt-%, or from 5 to 90 wt-%, from 25 to 70 wt-%, or from 40 to 60 wt-% of the film delivery system are dissolved after 30 minutes as measured using a USP apparatus 1 (basket) at 37±0.5° C. in 900 mL 0.01 N HCl.

In particular embodiments, the film delivery system according to the present invention is for the administration of an active ingredient. In certain further embodiments, the film delivery system is for the oral and/or transmucosal administration of an active ingredient. In certain embodiments, the invention is also related to a method of administration, and in particular of an oral and/or transmucosal administration of an active ingredient by administering the film delivery system according to the invention to a human patient. In certain embodiments, the invention relates to the use of the inventive film delivery system for the manufacture of a medicament for administration, and in particular of an oral and/or transmucosal administration of an active ingredient.

### Process of Manufacture

The invention further relates to a process of manufacture of a film delivery system, and in particular of the inventive film delivery system as outlined above.

In such a process of manufacture, the mucoadhesive layer is prepared in a first step i) and then provided with the perforations in a second step ii), thus, the inventive process of manufacture of a film delivery system comprises a mucoadhesive layer, comprising the steps of:
i) preparing a mucoadhesive layer; and
ii) providing the mucoadhesive layer with 5 to 100 perforations per cm² by using a perforating tool.

The mucoadhesive layer can be prepared in any conventional manner known and in particular by a hot-melt process or by a film casting process. Polymers for hot-melt manufacturing processes can be more rigid and often require a certain thickness of the film (then also resulting in more rigid films), e.g., in order to be able to incorporate a sufficient amount of active ingredient, and/or to ensure the necessary processability. However, thicker and/or more rigid films are disadvantageous in terms of haptics and by potentially causing discomfort to the patient. The inventive film delivery systems as explained further above provide for an improved mouthfeel which makes up for the potentially rigid and thicker films of hot melt processable polymers and thus have a larger choice of possible hot-melt materials.

Any conventional hot-melt process such as a vacuum compression molding process commonly used for laboratory scale as well as a hot-melt extrusion process which is advantageous for large scale production can be used. Thus, in certain embodiments, in step i) of the process of manufacture, the mucoadhesive layer is prepared by a hot-melt process, and the hot-melt process is

a hot-melt extrusion process comprising the steps of:
a) Introducing a film-forming agent or a mixture comprising a film-forming agent into an extruder;
b) heating said film-forming agent or said mixture to at least the softening temperature of the film-forming agent or of said mixture; and
c) extruding the heated film-forming agent or the heated mixture comprising the film-forming agent in the form a film to obtain the mucoadhesive layer
   or a vacuum compression molding process comprising the steps of
a) Introducing a film-forming agent or a mixture comprising a film-forming agent into a sample chamber; and
b) Compacting said film-forming agent or said mixture by moving down a piston while applying vacuum and heating said film-forming agent or said mixture to at least the softening temperature of the film-forming agent or of said mixture to obtain the mucoadhesive layer.

The mucoadhesive layer can of course also be prepared by a conventional film casting process that is adequate for most polymers. Thus, in certain embodiments, in step i) of the process of manufacture, the mucoadhesive layer is prepared by a film casting process, and the film casting process comprises the steps of.
a) combining a film-forming agent and a solvent to obtain a coating composition;
b) coating the coating composition; and
c) drying the coated coating composition to form the mucoadhesive layer.

In step ii) the perforations are provided by employing a perforating tool. As outlined above, in its simplest form, a film delivery system consists of a mucoadhesive layer and then only the mucoadhesive layer has to be provided with a perforation. Where the film delivery system consists of more layers, the perforations can be provided to the whole film through all layers, or to at least part of the layers and more than to only the mucoadhesive layer.

On the other hand, in certain embodiments, the mucoadhesive layer could be provided with perforations and thereafter laminated or otherwise adhered to the remaining layers, to thus obtain a film delivery system wherein only the mucoadhesive layer is provided with perforations. Such film delivery systems could be also prepared by controlled-depth piercing only the mucoadhesive film of a multi-layered film delivery system.

In any case, the perforations provide the mucoadhesive layer with at least one side (the pierced or the piercing side) covered by openings. It is this piercing side, or the side covered by openings, that is intended to be placed on the mucosa (i.e., it is a mucosa-contacting side).

Exemplary perforating tools are cutting devices, needle devices and punching devices, i.e., in certain embodiments, in step ii), the mucoadhesive layer is provided with perforations by using a perforating tool selected from cutting devices, needle devices and punching devices.

In case in step ii), the mucoadhesive layer is provided with perforations by piercing the mucoadhesive layer with a perforating tool selected from needle devices, these can be selected from simple needles, needle assemblies, i.e., a plurality of needles assembled in one device so that a plurality of perforations can be prepared at the same time, and pin rollers, i.e., a roller equipped with a plurality of pins for piercing, which can be installed in a manufacturing line for large-scale production processes. Such needle devices may be provided with needles or pins having a diameter of at least 0.1, 0.2, 0.3 or 0.5 mm, or of 3 mm or less, 2 mm or less, or 1 mm or less, and may have a blunt tip or a sharp needle head.

When providing perforations that span the whole thickness of the mucoadhesive layer (e.g., through-holes or full slits) with such needle devices, it is possible that by piercing with a tip or a needle head, the material of the pierced layer is pushed out at the opposite side and forms fringes resulting in a frayed edge (FIG. 7d). In such a case, in view of an improved haptics, it is the piercing side without the fringes that is preferably applied to the mucosa.

In case a punching device is used as a perforating tool, in step ii), the mucoadhesive layer is provided with perforations by punching the mucoadhesive layer with such a perforating tool selected from punching devices, thereby removing the punched part from the mucoadhesive layer. Using such punching devices will avoid the formation of fringes which is believed to be advantageous for an improved haptic. On the other hand, punching will remove a part from the mucoadhesive layer and thus reduce the amount of active ingredient contained, so from that point of view, piercing or cutting may be more preferable.

### EXAMPLES

The present invention will now be more fully described with reference to the accompanying examples. It should be understood, however, that the following description is illustrative only and should not be taken in any way as a restriction of the invention. Numerical values provided in the examples regarding the amount of ingredients in the composition or the area weight may vary slightly due to manufacturing variability, which, however, does not affect the advantages provided by the invention.

### Example 1

### Preparation of Mucoadhesive Layer Samples

The formulation and perforation patterns, as well as the area weight of the muco-adhesive layer used for the Comparison Experiments 1a to 1e are summarized in Table 1.1 below.

TABLE 1.1

| Ingredient | Amount | Perforation | Area weight |
|---|---|---|---|
| (Trade Name) | [wt-%] | patterns | [g/m²] |
| Hydroxypropyl | 100 | Non-perforated, A, B, C | 300 |
| cellulose | | | |
| (Klucel EF) | | | |

The polymer hydroxypropyl cellulose (Klucel EF, molecular weight: 80,000) was filled inside the sample holder of a MeltPrep® vacuum compression molding (VCM) device from MeltPrep GmbH consisting of the sample holder connected to a vacuum source, a piston and a lid. The sample material was heated to 160° C. using a hot plate heating unit, vacuum was applied, and the piston moved down for compacting the sample and preventing bubbles. The obtained homogenous disc was cooled by convection air cooling to provide a mucoadhesive layer sample with a diameter of 2.5 cm and an area of 4.91 cm².

### Perforation Patterns (Concerning all Examples)

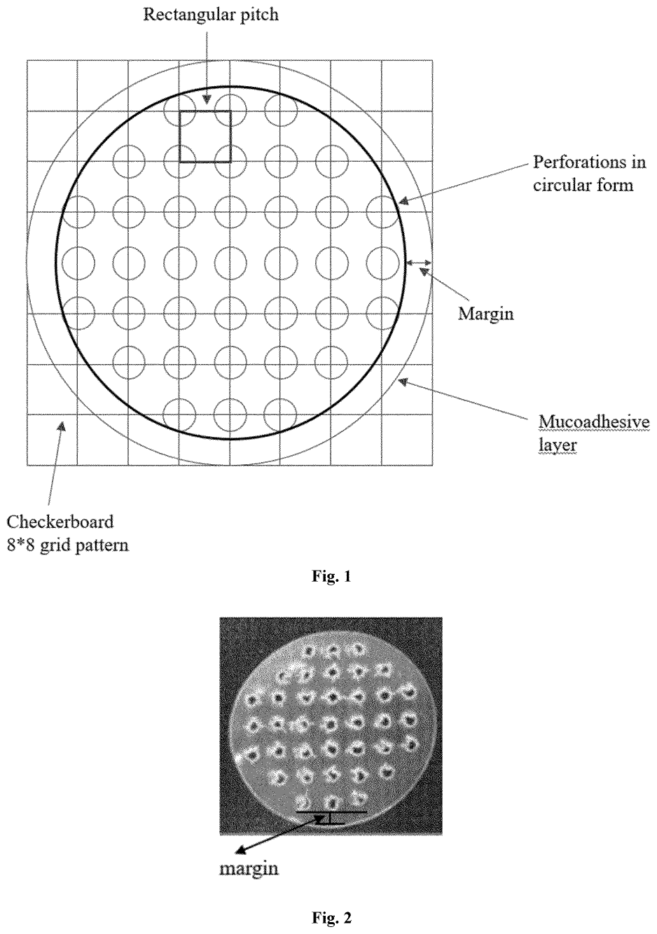

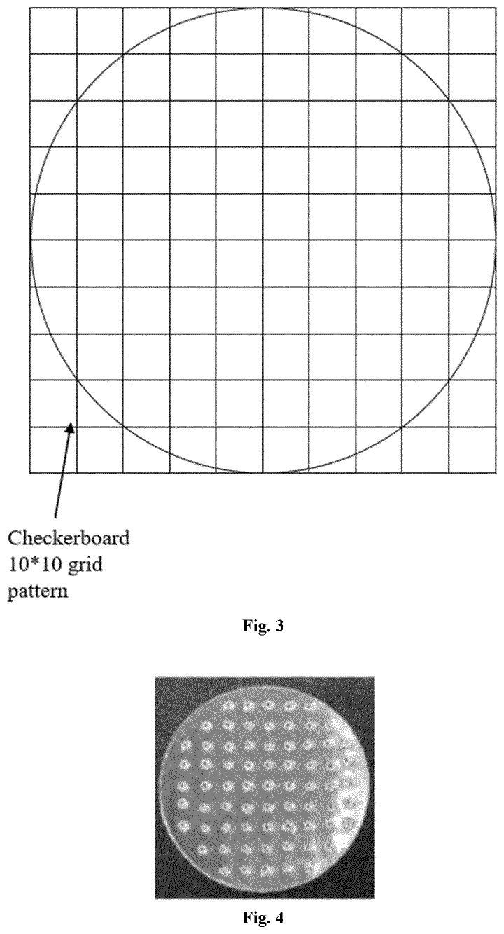

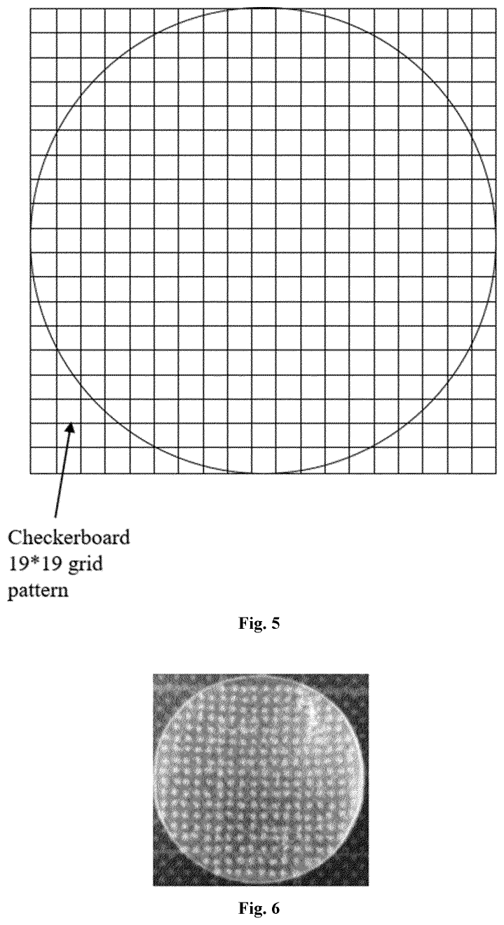

Perforation of the mucoadhesive layer was performed using needles according to one of three perforation patterns (A, B, C), with the needle diameter and number of perforations being varied. For each perforation pattern, a template provided with a checkerboard grid pattern was prepared (see FIG. 1) and mounted on a black foamed plastic plate (pattern A) or on a double-folded cellulose sheet (patterns B and C). The mucoadhesive layer sample was affixed on the template and manually pierced at each intersection point so as to reach the maximum possible piercing depth, thus creating perforations with a circular form of uniform geometry. Tables 1.2 and 1.3 below provide a summary of the pattern (Table 1.2) and perforation specifics (Table 1.3).

TABLE 1.2

| | | Sample size | Number of perforations | |
|---|---|---|---|---|
| Pattern | | Total area | Per sample | Per total area |
| | Grid | [cm²] | [number] | [number/cm²] |
| A | 8*8 | 4.91 | 37 | 7.5 |
| | (pitch: 3.125 mm) | | | |
| B | 10*10 | 4.91 | 69 | 14.1 |
| | (pitch: 2.5 mm) | | | |
| C | 19*19 | 4.91 | 276 | 56.2 |
| | (pitch: 1.316 mm) | | | |

TABLE 1.3

| | Perforation | Perforation Perimeter** | | Perforation area** | |
|---|---|---|---|---|---|
| | Diameter* | Per perf. | Per total area | Per perf. | Per total area |
| Pattern | [mm] | [cm/perf.] | [cm/cm²] | [cm²/perf.] | [% cm²/cm²] |
| A | 2 | 0.63 | 4.71 | 0.0314 | 23.7 |
| B | 1 | 0.31 | 4.43 | 0.00785 | 11.0 |
| C | 0.5 | 0.16 | 8.83 | 0.00196 | 11.0 |

*Maximum needle diameter
**calculated as perimeter / area of a circle based on maximum needle diameter

### Preparation of the Film Delivery System (Concerning all Examples)

In the present case, the mucoadhesive layer discs correspond to the individual (placebo) film delivery systems. In specific embodiments, such a film delivery system can be provided with further layers such as a cosmetic layer and/or a backing layer. The film delivery systems are then sealed into pouches of the primary packaging material as conventional in the art.

### Evaluation of Samples: Comparison Experiments 1a to 1e

The individual film delivery systems were evaluated in terms of initial mouthfeel, fast attachment on mucosa/less foreign body sensation and dissolution behavior by applying one film delivery system to each of the left and the right buccal mucosa, to thus compare the performance of the two administered systems as shown in Table 1.4. Preliminary experiments had been conducted to demonstrate that the results are independent from the application side, i.e., it was immaterial for the evaluation which of the two compared systems was applied to the left or right administration site.

TABLE 1.4

| | | | Performance mouthfeel | | Performance |
|---|---|---|---|---|---|
| Comparison of different | | | | Fast attachment / | dissolution |
| patterns | | | Initial | less foreign | Faster |
| Exp. | Pattern 1 | Pattern 2 | mouthfeel | body sensation | dissolution |
| 1a | Non-perf. | A | N/A | A > Non-Perf. | A > Non-Perf. |
| 1b | Non-perf. | B | B > Non-Perf. | B > Non-Perf. | B > Non-Perf. |
| 1c | Non-perf. | C | C > Non-Perf. | C > Non-Perf. | C > Non-Perf. |
| 1d | A | B | B > A | B > A | N/A |
| 1e | B | C | C > B | C > B | N/A |

* In the above table, “>” indicates the superior performance, i.e., where B > Non-Perf., the performance of the system with pattern B was superior to the non-perforated system

Comparisons 1a to 1c show a faster attachment on the mucosa, less foreign body sensation and a faster dissolution (as observed by less residual material remaining on the mucosa) for the systems provided with perforations compared to the non-perforated systems of the same formulation. The systems with perforation patterns B and C additionally show an advantageous initial mouthfeel compared to the non-perforated system. Comparison Experiments 1d and 1e show that a higher number of perforations is beneficial for the mouthfeel performance, both in terms of initial mouthfeel as well as attachment on the mucosa and foreign body sensation.

### Example 2

### Preparation of Mucoadhesive Layer Samples

The formulation and perforation patterns, as well as the area weight of the muco-adhesive layer used for the Comparison Experiments 2a to 2c are summarized in Table 2.1 below.

TABLE 2.1

| Ingredient | Amount | Perforation | Area weight |
|---|---|---|---|
| (Trade Name) | [wt-%] | patterns | [g/m²] |
| Hydroxypropyl | 90 | Non-perforated, B, C | 300 |
| cellulose | | | |
| (Klucel EF) | | | |
| Sorbitol | 10 | | |

The polymer hydroxypropyl cellulose (Klucel EF, molecular weight: 80,000) was mixed with the sorbitol and pulverized using a mortar and pestle. The resulting mixture was filled inside the sample holder of a MeltPrep® VCM device and a mucoadhesive layer sample was produced in the same way as described in Example 1.

### Perforation Patterns

Perforation of the mucoadhesive layer was performed using needles according to one of two perforation patterns (B, C), as described in Example 1 (see tables 1.2 and 1.3 above for a summary of the pattern and perforation specifics).

### Evaluation of Samples: Comparison Experiments 2a to 2c

The individual (placebo) film delivery systems (see Example 1 for the preparation of the systems) were evaluated in the same way as described in Example 1, to thus compare the performance of the two administered systems as shown in Table 2.2 below.

TABLE 2.2

| | | | Performance mouthfeel | | Performance |
|---|---|---|---|---|---|
| Comparison of different | | | | Fast attachment / | dissolution |
| patterns | | | Initial | less foreign | Faster |
| Exp. | Pattern 1 | Pattern 2 | mouthfeel | body sensation | dissolution |
| 2a | Non-perf. | B | B > Non-Perf. | B > Non-Perf. | B > Non-Perf. |
| 2b | Non-perf. | C | C > Non-Perf. | C > Non-Perf. | C > Non-Perf. |
| 2c | B | C | C > B | C > B | N/A |

* In the above table, “>” indicates the superior performance, i.e., where B > Non-Perf., the performance of the system with pattern B was superior to the non-perforated system

Comparisons 2a and 2b show a faster attachment on the mucosa, less foreign body sensation and a faster dissolution (as observed by less residual material remaining on the mucosa) for the systems provided with perforations compared to the non-perforated systems of the same formulation. The systems with perforation patterns B and C additionally show an advantageous initial mouthfeel compared to the non-perforated system. Comparison Experiment 2c shows that a higher number of perforations is beneficial for the mouthfeel performance, both in terms of initial mouthfeel as well as attachment on the mucosa and foreign body sensation. This is particularly beneficial for any relatively rigid film.

### Example 3

### Preparation of Mucoadhesive Layer Samples

The formulation and perforation patterns, as well as the area weight of the muco-adhesive layer used for Comparison Experiment 3 are summarized in Table 3.1 below.

TABLE 3.1

| Ingredient | Amount | Perforation | Area weight |
|---|---|---|---|
| (Trade Name) | [wt-%] | patterns | [g/m²] |
| Hydroxypropylmethyl | 100 | Non-perforated, B | 300 |
| cellulose | | | |
| (Affinisol HME, 15LV) | | | |

The polymer hydroxypropylmethyl cellulose (Affinisol HME 15 LV, molecular weight: 85,000) was filled inside the sample holder of a MeltPrep® VCM device and a mucoadhesive layer sample was produced in the same way as described in Example 1, except that the sample material was heated to 250° C.

### Perforation Patterns

Perforation of the mucoadhesive layer was performed using needles according to perforation pattern B as described in Example 1 (see tables 1.2 and 1.3 above for a summary of the pattern and perforation specifics).

### Evaluation of Samples: Comparison Experiment 3

The individual (placebo) film delivery systems (see Example 1 for the preparation of the systems) were evaluated in the same way as described in Example 1, to thus compare the performance of the two administered systems as shown in Table 3.2 below.

TABLE 3.2

| | | | Performance mouthfeel | | Performance |
|---|---|---|---|---|---|
| Comparison of different | | | | Fast attachment / | dissolution |
| patterns | | | Initial | less foreign | Faster |
| Exp. | Pattern 1 | Pattern 2 | mouthfeel | body sensation | dissolution |
| 3 | Non-perf. | B | B > Non-Perf. | B > Non-Perf. | B > Non-Perf. |

* In the above table, “>” indicates the superior performance, i.e., where B > Non-Perf., the performance of the system with pattern B was superior to the non-perforated system

Comparison Experiment 3 shows a superior performance for all mouthfeel and dissolution evaluations for the system provided with perforations compared to the non-perforated systems of the same formulation.

### Example 4

### Preparation of Mucoadhesive Layer Samples

The formulation and perforation patterns, as well as the area weight of the muco-adhesive layer used for Comparison Experiment 4 are summarized in Table 4.1 below.

TABLE 4.1

| Ingredient | Amount | Perforation | Area weight |
|---|---|---|---|
| (Trade Name) | [wt-%] | patterns | [g/m²] |
| Polyethylene oxide | 100 | Non-perforated, B | 300 |
| (Polyox N10) | | | |

The polymer polyethylene oxide (Polyox N10, molecular weight: 100,000) was filled inside the sample holder of a MeltPrep® VCM device and a mucoadhesive layer sample was produced in the same way as described in Example 1, except that the sample material was heated 10 to 130° C.

### Perforation Patterns

Perforation of the mucoadhesive layer was performed using needles according to perforation pattern B as described in Example 1 (see tables 1.2 and 1.3 above for a summary of the pattern and perforation specifics).

### Evaluation of Samples: Comparison Experiment 4

The individual (placebo) film delivery systems (see Example 1 for the preparation of the systems) were evaluated in the same way as described in Example 1, to thus compare the performance of the two administered systems as shown in Table 4.2 below.

TABLE 4.2

| | | | Performance mouthfeel | | Performance |
|---|---|---|---|---|---|
| Comparison of different | | | | Fast attachment / | dissolution |
| patterns | | | Initial | less foreign | Faster |
| Exp. | Pattern 1 | Pattern 2 | mouthfeel | body sensation | dissolution |
| 4 | Non-perf. | B | B > Non-Perf. | B > Non-Perf. | B > Non-Perf. |

* In the above table, “>” indicates the superior performance, i.e., where B > Non-Perf., the performance of the system with pattern B was superior to the non-perforated system

Comparison Experiment 4 shows a superior performance for all mouthfeel and dissolution evaluations for the system provided with perforations compared to the non-perforated systems of the same formulation.

### Example 5

### Preparation of Mucoadhesive Layer Samples

The formulation and perforation patterns, as well as the area weight of the muco-adhesive layer used for the Comparison Experiments 5a to 5e are summarized in Table 5.1 below.

TABLE 5.1

| Ingredient | Amount | Perforation | Area weight |
|---|---|---|---|
| (Trade Name) | [wt-%] | patterns | [g/m²] |
| Polyethylene oxide | 100 | Non-perforated, B | 300 |
| (Polyox N80) | | | |

The polymer polyethylene oxide (Polyox N80, molecular weight: 200,000) was filled inside the sample holder of a MeltPrep® VCM device and a mucoadhesive layer sample was produced in the same way as described in Example 1, except that the sample material was heated 130° C.

### Perforation Patterns

Perforation of the mucoadhesive layer was performed using needles according to one of three perforation patterns (A, B, C), as described in Example 1 (see tables 1.2 and 1.3 above for a summary of the pattern and perforation specifics).

### Evaluation of Samples: Comparison Experiments 5a to 5e

The individual (placebo) film delivery systems (see Example 1 for the preparation of the systems) were evaluated in the same way as described in Example 1, to thus compare the performance of the two administered systems as shown in Table 5.2 below.

TABLE 5.2

| | | | Performance mouthfeel | | Performance |
|---|---|---|---|---|---|
| Comparison of different | | | | Fast attachment / | dissolution |
| patterns | | | Initial | less foreign | Faster |
| Exp. | Pattern 1 | Pattern 2 | mouthfeel | body sensation | dissolution |
| 5a | Non-perf. | A | N/A | A > Non-Perf. | A > Non-Perf. |
| 5b | Non-perf. | B | B > Non-Perf. | B > Non-Perf. | B > Non-Perf. |
| 5c | Non-perf. | C | C > Non-Perf. | C > Non-Perf. | C > Non-Perf. |
| 5d | A | B | B > A | B > A | N/A |
| 5e | B | C | C > B | C > B | N/A |

* In the above table, “>” indicates the superior performance, i.e., where B > Non-Perf., the performance of the system with pattern B was superior to the non-perforated system

Comparisons 5a to 5c show a faster attachment on the mucosa, less foreign body sensation and a faster dissolution (as observed by less residual material remaining on the mucosa) for the systems provided with perforations compared to the non-perforated systems of the same formulation. The systems with perforation patterns B and C additionally show an advantageous initial mouthfeel compared to the non-perforated system. Comparison Experiments 5d and 5e show that a higher number of perforations is beneficial for the mouthfeel performance, both in terms of initial mouthfeel as well as attachment on the mucosa and foreign body sensation.

### Example 6

### Preparation of Mucoadhesive Layer Samples

The formulation and perforation patterns, as well as the area weight of the muco-adhesive layer used for Comparison Experiment 6 are summarized in Table 6.1 below.

TABLE 6.1

| Ingredient | Amount | Perforation | Area weight |
|---|---|---|---|
| (Trade Name) | [wt-%] | patterns | [g/m²] |
| Polyvinyl alcohol | 90 | Non-perforated, B | 300 |
| (Parteck ® MXP 4-88) | | | |
| Sorbitol | 10 | | |

The polymer polyvinyl alcohol (Parteck® MXP 4-88, molecular weight: 32,000) was mixed with the sorbitol and pulverized using a mortar and pestle. The resulting mixture was filled inside the sample holder of a MeltPrep® VCM device and a mucoadhesive layer sample was produced in the same way as described in Example 1, except that the sample material was heated to 230° C.

### Perforation Patterns

Perforation of the mucoadhesive layer was performed using needles according to perforation pattern B as described in Example 1 (see tables 1.2 and 1.3 above for a summary of the pattern and perforation specifics).

### Evaluation of Samples: Comparison Experiment 6

The individual (placebo) film delivery systems (see Example 1 for the preparation of the systems) were evaluated in the same way as described in Example 1, to thus compare the performance of the two administered systems as shown in Table 6.2 below.

TABLE 6.2

| | | | Performance mouthfeel | | Performance |
|---|---|---|---|---|---|
| Comparison of different | | | | Fast attachment / | dissolution |
| patterns | | | Initial | less foreign | Faster |
| Exp. | Pattern 1 | Pattern 2 | mouthfeel | body sensation | dissolution |
| 6 | Non-perf. | B | B > Non-Perf. | B > Non-Perf. | B > Non-Perf. |

* In the above table, “>” indicates the superior performance, i.e., where B > Non-Perf., the performance of the system with pattern B was superior to the non-perforated system

Comparison Experiment 6 shows a superior performance for all mouthfeel and dissolution evaluations for the system provided with perforations compared to the non-perforated systems of the same formulation.

The invention relates in particular to the following further items:

1. Film delivery system comprising a mucoadhesive layer, wherein
the mucoadhesive layer comprises a film-forming agent and is provided with 5 to 100 perforations per cm².
   2. Film delivery system according to item 1,
wherein the film delivery system has a total weight of at least 5 mg or 10 mg, of less than or equal to 175 mg or 150 mg, or has a total weight of from 5 to 175 mg or from 10 to 150 mg.
   3. Film delivery system according to item 2,
wherein the mucoadhesive layer is provided with at least 7, at least 10, at least 20 or at least 50 perforations per cm² or with less than 90, less than 80 or less than 60 perforations per cm².
   4. Film delivery system according to any one of items 1 to 3,
wherein the perforations are in the form of a hole, and in particular a through-hole or a pocket-hole, or in the form of a slit, and in particular a full slit or an incomplete slit, and/or
wherein the perforations have a circular, an oval, a triangular, a rectangular or a hexagonal form.
   5. Film delivery system according to according to any one of items 1 to 4,
Wherein the perforations have a surface area of at least 0.0005 cm², at least 0.001 cm² or at least 0.002 cm² per perforation, or a surface area per area of the mucoadhesive layer of at least 2%, at least 5% or at least 10%.
   6. Film delivery system according to according to any one of items 1 to 5,
wherein the perforations have a surface area of 0.15 cm² or less, 0.05 cm² or less or 0.03 cm² or less per perforation, or a surface area per area of the mucoadhesive layer of 70% or less, 50% or less or 30% or less.
   7. Film delivery system according to any one of items 1 to 6,
wherein the perforations have a perimeter of at least 0.1 cm, at least 0.2 cm or at least 0.3 cm per perforation, or a perimeter per area of the mucoadhesive layer of at least 2 cm/cm², at least 4 cm/cm² or at least 8 cm/cm².
   8. Film delivery system according to any one of items 1 to 7,
Wherein the perforations have a perimeter of 2 cm or less, 1 cm or less or 0.8 cm or less per perforation, or a perimeter per area of the mucoadhesive layer of 30 cm/cm² or less, 20 cm/cm² or less or 15 cm/cm² or less.
   9. Film delivery system according to any one of items 1 to 8,
wherein the perforations all have the same form, or two or more different forms.
   10. Film delivery system according to any one of items 1 to 9,
wherein the perforations are distributed evenly or non-evenly.
   11. Film delivery system according to item 10,
wherein the perforations are distributed evenly with a triangular, a rectangular, a staggered or a diagonal pitch.
   12. Film delivery system according to any one of items 1 to 11,
wherein the mucoadhesive layer has a margin, which is free of perforations, or has no margin.
   13. Film delivery system according to any one of items 1 to 12,
wherein the perforations are incorporated by using a perforating tool.
   14. Film delivery system according to item 13,
wherein the perforating tool is selected from cutting devices, needle devices and punching devices.
   15. Film delivery system according to item 14,
wherein the needle device is selected from the group consisting of needles, needle assemblies, and pin rollers.
   16. Film delivery system according to item 14 or 15,
wherein the needle device is provided with needles or pins having a diameter of at least 0.1, 0.2, 0.3 or 0.5 mm.
   17. Film delivery system according to any one of items 14 to 16,
wherein the needle device is provided with needles or pins having a diameter of 3 mm or less, 2 mm or less, or 1 mm or less.
   18. Film delivery system according to any one of items 14 to 17,
wherein the needle device has a blunt tip or a sharp needle head.
   19. Film delivery system according to item 14,
wherein the cutting device cuts a perforation in the form of a full slit by full-depth cutting, or a perforation in the form of an incomplete slit by controlled-depth cutting,
wherein the needle device incorporates a perforation in the form of a through-hole by piercing through the mucoadhesive layer, or a perforation in the form of a pocket-hole by controlled-depth piercing the mucoadhesive layer, and
wherein the punching device punches a perforation and thereby removes the punched part from the mucoadhesive layer.
   20. Film delivery system according to any one of items 1 to 19,
wherein the mucoadhesive layer is obtainable by a hot-melt process or by a film casting process.
   21. Film delivery system according to any one of items 1 to 20,
wherein the film-forming agent is a dissolvable film-forming agent.
   22. Film delivery system according to any one of items 1 to 21,
wherein the film-forming agent is a polymer, a natural film-forming agent, or any mixture thereof.
   23. Film delivery system according to any one of items 1 to 22,
wherein the film-forming agent is
   a polymer selected from the group consisting of polyvinylpyrrolidone, methyl cellulose, ethyl cellulose, hydroxyethyl cellulose, hydroxypropyl cellulose, hydroxypropylmethyl cellulose, carboxymethyl cellulose sodium, polyethylene glycol-polyvinyl acetate- and polyvinylcaprolactame-based graft copolymers, polyvinyl alcohol, polyvinyl alcohol-polyethylene glycol copolymers, polyvinylpyrrolidone-polyvinylacetate copolymers, polyethylene oxides, polyethylene glycols, methacrylic acid-methyl methacrylate copolymers, and methacrylic acid-ethyl methacrylate copolymers, or
   a natural film-forming agent selected from the group consisting of shellac, pectin, gelatine, alginate, pullulan and starch derivatives, or
   any mixture thereof.
      24. Film delivery system according to item 23,
wherein the film-forming agent is selected from the group consisting of hydroxypropyl cellulose, hydroxypropylmethyl cellulose, polyethylene oxide, polyvinyl alcohol, and any mixture thereof.
   25. Film delivery system according to item 24,
wherein the film-forming agent is a hydroxypropyl cellulose.
   26. Film delivery system according to item 25,
wherein the film-forming agent is a hydroxypropyl cellulose having a molecular weight of from 30,000 to 1,500,000, or from 30,000 to 500,000, or any mixture thereof.
   27. Film delivery system according to item 26,
wherein the film-forming agent is a hydroxypropyl cellulose having a molecular weight of 80,000, 95,000 or 370,000, or any mixture thereof.
   28. Film delivery system according to item 24,
wherein the film-forming agent is a hydroxypropylmethyl cellulose.
   29. Film delivery system according to item 24,
wherein the film-forming agent is a hydroxypropylmethyl cellulose having a molecular weight of 85,000, 180,000 or 550,000, or any mixture thereof.
   30. Film delivery system according to item 24,
wherein the film-forming agent is a polyethylene oxide.
   31. Film delivery system according to item 30,
wherein the film-forming agent is a polyethylene oxide having a molecular weight in the range of from 100,000 to 7,000,000, or any mixture thereof.
   32. Film delivery system according to item 31,
wherein the film-forming agent is a polyethylene oxide having a molecular weight selected from the group consisting of 100,000, 200,000, 300,000, 600,000, 900,000, 1,000,000, 2,000,000, 4,000,000, 5,000,000 and 7,000,000, or any mixture thereof.
   33. Film delivery system according to item 32,
wherein the film-forming agent is a polyethylene oxide having a molecular weight of 100,000 or 200,000, or a mixture thereof.
   34. Film delivery system according to item 24,
wherein the film-forming agent is a polyvinyl alcohol.
   35. Film delivery system according to item 34,
wherein the film-forming agent is a polyvinyl alcohol having a molecular weight in the range of from 20,000 to 150,000, or any mixture thereof.
   36. Film delivery system according to item 35,
wherein the film-forming agent is a polyvinyl alcohol having a molecular weight of 32,000.
   37. Film delivery system according to any one of items 1 to 36,
wherein the amount of the film-forming agent is
   at least 40 wt-%, at least 70 wt-%, or at least 80 wt-%,
   100 wt-% or less, or 95 wt-% or less, or is
   100 wt-% or 90 wt-%
of the mucoadhesive layer.
   38. Film delivery system according any one of items 1 to 37,
wherein the mucoadhesive layer further comprises one or more excipients selected from the group consisting of fatty acids, sweeteners, flavoring agents, colorants, permeation enhancers, solubilizers, plasticizers, humectants, disintegrants, wetting enhancers, dissolution enhancers, emulsifiers, antioxidants, stabilizers, buffer reagents and further film-forming agents.
   39. Film delivery system according to item 38,
wherein the mucoadhesive layer further comprises a plasticizer selected from the group consisting of mono-, di-, oligo- and polysaccharides and derivatives such as sorbitol, polyethylene glycol, triacetin, triethyl citrate, propylene glycol, glycerol and medium chain triglycerides.
   40. Film delivery system according to item 39,
wherein the mucoadhesive layer further comprises
   a fatty acid selected from the group consisting of a saturated or unsaturated, linear or branched carboxylic acid comprising 4 to 24 carbon atoms, and in particular is selected from the group consisting of caprylic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid, and/or
   one or more natural or artificial sweeteners selected from the group consisting of saccharose, glucose, fructose, sorbitol, mannitol, isomalt, maltitol, lactitol, xylitol, erythritol, sucralose, acesulfame potassium, aspartame, cyclamate, neohesperidine, neotame, steviol glycosides, thaumatin and saccharin sodium, and/or
   one or more natural or artificial flavoring agents selected from the group consisting of vanillin, methyl salicylate, menthol, manzanate, diacetyl, acetylpropionyl, acetoin, isoamyl acetate, benzaldehyde, cinnamaldehyde, ethyl propionate, methyl anthranilate, limonene, ethyl decadienoate, allyl hexanoate, ethyl maltol, 2,4-dithiapentane, ethylvanillin and eucalyptol as well as flavoring compositions such as peppermint flavor.
      41. Film delivery system according to any one of items 1 to 40,
wherein the mucoadhesive layer has an area weight of at least 70 g/m², at least 100 g/m², or at least 200 g/m².
   42. Film delivery system according to any one of items 1 to 41,
wherein the mucoadhesive layer has an area weight of 700 g/m² or less, 500 g/m² or less, 400 g/m² or less or 300 g/m² or less.
   43. Film delivery system according to any one of items 1 to 42,
wherein the film delivery system comprises or does not comprise a cosmetic layer, and/or
wherein the film delivery system comprises or does not comprise a backing layer, and/or
wherein the film delivery system is provided with a means to indicate which side has to be applied to the mucosa, wherein in particular the means is a one-sided coloring or imprinting or is the presence of a detachable release liner.
   44. Film delivery system according to any one of items 1 to 43, which is a mucoadhesive delivery system.
   45. Film delivery system according to any one of items 1 to 44 for application to the mucosa, and in particular to the mucosa of the oral cavity of a human patient.
   46. Film delivery system according to item 45 for application to the buccal, sublingual, gingival or palatal mucosa, preferably the buccal mucosa of the oral cavity of a human patient.
   47. Film delivery system according to any one of items 1 to 46 for the administration of an active ingredient, and in particular for the oral and/or transmucosal administration of an active ingredient.
   48. Film delivery system according to any one of items 45 to 47,
wherein the film delivery system is administered by applying the film to and maintained on the mucosa for the administration period, after which the film delivery system is dissolved, eroded or substantially disintegrated, or is removed from the application site, and/or
wherein at least 5 wt-%, 25 wt-% or 40 wt-%, or less than 90 wt-%, 70 wt-% or 60 wt-%, or from 5 to 90 wt-%, from 25 to 70 wt-%, or from 40 to 60 wt-% of the film delivery system are dissolved after 5 minutes as measured using a USP apparatus 1 (basket) at 37±0.5° C. in 900 mL 0.01 N HCl.
   49. Film delivery system according to any one of items 1 to 48 for use in a method of treating a human patient.
   50. A method of treatment,
wherein the film delivery system according to any one of items 1 to 49 is administered to a human patient.
   51. Use of a film delivery system according to any one of items 1 to 49 for the manufacture of a medicament for treating a human patient.
   52. Process of manufacture of a film delivery system comprising a mucoadhesive layer, comprising the steps of:
i) preparing a mucoadhesive layer; and
ii) providing the mucoadhesive layer with 5 to 100 perforations per cm² by using a perforating tool.
   53. Process according to item 52, wherein, in step i), the mucoadhesive layer is prepared by a hot-melt process or by a film casting process.
   54. Process according to item 53, wherein
in step i), the mucoadhesive layer is prepared by a hot-melt process, and the hot-melt process is
a hot-melt extrusion process comprising the steps of:
   a) Introducing a film-forming agent or a mixture comprising a film-forming agent into an extruder;
   b) heating said film-forming agent or said mixture to at least the softening temperature of the film-forming agent or of said mixture; and
   c) extruding the heated film-forming agent or the heated mixture comprising the film-forming agent in the form a film to obtain the mucoadhesive layer
or a vacuum compression molding process comprising the steps of
   a) Introducing a film-forming agent or a mixture comprising a film-forming agent into a sample chamber; and
   b) Compacting said film-forming agent or said mixture by moving down a piston while applying vacuum and heating said film-forming agent or said mixture to at least the softening temperature of the film-forming agent or of said mixture to obtain the mucoadhesive layer.
      55. Process according to item 53, wherein
in step i), the mucoadhesive layer is prepared by a film casting process, and the film casting process comprises the steps of
   a) combining a film-forming agent and a solvent to obtain a coating composition;
   b) coating the coating composition; and
   c) drying the coated coating composition to form the mucoadhesive layer.
      56. Process according to any one of items 52 to 55, wherein
in step ii), the mucoadhesive layer is provided with perforations by using a perforating tool selected from cutting devices, needle devices and punching devices.
   57. Process according to item 56, wherein
in step ii), the mucoadhesive layer is provided with perforations by piercing the mucoadhesive layer with a perforating tool selected from needle devices and
the needle device is selected from the group consisting of needles, needle assemblies, and pin rollers.
   58. Process according to item 57, wherein
the needle device is provided with needles or pins having a diameter of at least 0.1, 0.2, 0.3 or 0.5 mm.
   59. Process according to item 57 or 58, wherein
the needle device is provided with needles or pins having a diameter of 3 mm or less, 2 mm or less, or 1 mm or less.
   60. Process according to item any one of items 57 to 59, wherein
the needle device has a blunt tip or a sharp needle head.
   61. Process according to item 56, wherein
in step ii), the mucoadhesive layer is provided with perforations by punching the mucoadhesive layer with a perforating tool selected from punching devices, thereby removing the punched part from the mucoadhesive layer.
   62. Film delivery system comprising a mucoadhesive layer, wherein
the mucoadhesive layer comprises a dissolvable film-forming agent and is provided with at least 10 to less than 20 perforations per cm²,
the perforations have a surface area per area of the mucoadhesive layer of at least 10% and 30% or less,
the perforations have a perimeter per area of the mucoadhesive layer of at least 4 cm/cm² and 15 cm/cm² or less,
the perforations all have a circular form and are distributed evenly, and
the mucoadhesive layer has an area weight of at least 200 g/m² and 400 g/m² or less.
   63. Film delivery system comprising a mucoadhesive layer, wherein
the mucoadhesive layer comprises a dissolvable film-forming agent and is provided with at least 50 to less than 60 perforations per cm²,
the perforations have a surface area per area of the mucoadhesive layer of at least 5% and 30% or less,
the perforations have a perimeter per area of the mucoadhesive layer of at least 8 cm/cm² and 15 cm/cm² or less,
the perforations all have a circular form and are distributed evenly, and
the mucoadhesive layer has an area weight of at least 200 g/m² and 400 g/m² or less.
   64. Film delivery system comprising a mucoadhesive layer, wherein
the film delivery system has a total weight of from 5 to 175 mg,
the mucoadhesive layer comprises a dissolvable film-forming agent and is provided with at least 10 to less than 20 perforations per cm²,
the perforations have a surface area per area of the mucoadhesive layer of at least 10% and 30% or less,
the perforations have a perimeter per area of the mucoadhesive layer of at least 4 cm/cm² and 15 cm/cm² or less,
the perforations all have a circular form and are distributed evenly, and
the mucoadhesive layer has an area weight of at least 200 g/m² and 400 g/m² or less.
   65. Film delivery system comprising a mucoadhesive layer, wherein
the film delivery system has a total weight of from 5 to 175 mg,
the mucoadhesive layer comprises a dissolvable film-forming agent and is provided with at least 50 to less than 60 perforations per cm²,
the perforations have a surface area per area of the mucoadhesive layer of at least 5% and 30% or less,
the perforations have a perimeter per area of the mucoadhesive layer of at least 8 cm/cm² and 15 cm/cm² or less,
the perforations all have a circular form and are distributed evenly, and
the mucoadhesive layer has an area weight of at least 200 g/m² and 400 g/m² or less.
   66. Film delivery system comprising a mucoadhesive layer, wherein
the mucoadhesive layer comprises a dissolvable film-forming agent and is provided with at least 10 to less than 20 perforations per cm²,
the perforations have a surface area per area of the mucoadhesive layer of at least 10% and 30% or less,
the perforations have a perimeter per area of the mucoadhesive layer of at least 4 cm/cm² and 15 cm/cm² or less,
the perforations all have a circular form and are distributed evenly, and
the mucoadhesive layer has an area weight of at least 200 g/m² and 300 g/m² or less.
   67. Film delivery system comprising a mucoadhesive layer, wherein
the mucoadhesive layer comprises a dissolvable film-forming agent and is provided with at least 50 to less than 60 perforations per cm²,
the perforations have a surface area per area of the mucoadhesive layer of at least 5% and 30% or less,
the perforations have a perimeter per area of the mucoadhesive layer of at least 8 cm/cm² and 15 cm/cm² or less,
the perforations all have a circular form and are distributed evenly, and
the mucoadhesive layer has an area weight of at least 200 g/m² and 300 g/m² or less.

1. Film delivery system comprising a mucoadhesive layer, wherein
the mucoadhesive layer comprises a film-forming agent and is provided with 5 to 100 perforations per cm².

2. Film delivery system according to claim 1,
wherein the film delivery system has a total weight of at least 5 mg or 10 mg, of less than or equal to 175 mg or 150 mg, or has a total weight of from 5 to 175 mg or from 10 to 150 mg.

3. Film delivery system according to claim 1 or 2,
wherein the mucoadhesive layer is provided with at least 7, at least 10, at least 20 or at least 50 perforations per cm², or with less than 90, less than 80 or less than 60 perforations per cm², and/or
wherein the perforations are in the form of a hole, and in particular a through-hole or a pocket-hole, or in the form of a slit, and in particular a full slit or an incomplete slit, and/or
wherein the perforations have a circular, an oval, a triangular, a rectangular or a hexagonal form.

4. Film delivery system according to according to any one of claims 1 to 3,
wherein the perforations have a surface area of at least 0.0005 cm², at least 0.001 cm² or at least 0.002 cm² per perforation, a surface area of 0.15 cm² or less, 0.05 cm² or less or 0.03 cm² or less per perforation, a surface area per area of the mucoadhesive layer of at least 2%, at least 5% or at least 10%, or a surface area per area of the mucoadhesive layer of 70% or less, 50% or less or 30% or less.

5. Film delivery system according to any one of claims 1 to 4,
wherein the perforations have a perimeter at least 0.1 cm, at least 0.2 cm or at least 0.3 cm per perforation, a perimeter of 2 cm or less, 1 cm or less or 0.8 cm or less per perforation, a perimeter per area of the mucoadhesive layer of at least 2 cm/cm², at least 4 cm/cm² or at least 8 cm/cm². or a perimeter per area of the mucoadhesive layer of 30 cm/cm² or less, 20 cm/cm² or less or 15 cm/cm² or less.

6. Film delivery system according to any one of claims 1 to 5,
wherein the perforations all have the same form, or two or more different forms, or are distributed evenly or non-evenly.

7. Film delivery system according to any one of claims 1 to 6,
wherein the film-forming agent is a dissolvable film-forming agent.

8. Film delivery system according to any one of claims 1 to 7,
wherein the film-forming agent is a polymer, a natural film-forming agent, or any mixture thereof, and preferably is
a polymer selected from the group consisting of polyvinylpyrrolidone, methyl cellulose, ethyl cellulose, hydroxyethyl cellulose, hydroxypropyl cellulose, hydroxypropylmethyl cellulose, carboxymethyl cellulose sodium, polyethylene glycol-polyvinyl acetate- and polyvinylcaprolactame-based graft copolymers, polyvinyl alcohol, polyvinyl alcohol-polyethylene glycol copolymers, polyvinylpyrrolidone-polyvinylacetate copolymers, polyethylene oxides, polyethylene glycols, methacrylic acid-methyl methacrylate copolymers, and methacrylic acid-ethyl methacrylate copolymers, or
a natural film-forming agent selected from the group consisting of shellac, pectin, gelatine, alginate, pullulan and starch derivatives, or
any mixture thereof.

9. Film delivery system according to any one of claims 1 to 8,
wherein the mucoadhesive layer has an area weight of at least 70 g/m², at least 100 g/m², or at least 200 g/m², or an area weight of 700 g/m² or less, 500 g/m² or less, 400 g/m² or less, or 300 g/m² or less.

10. Film delivery system according to any one of claims 1 to 9 for application to the mucosa, in particular to the mucosa of the oral cavity of a human patient, and preferably to the buccal, sublingual, gingival or palatal mucosa, preferably the buccal mucosa of the oral cavity of a human patient.

11. Film delivery system according to any one of claims 1 to 10 for the administration of an active ingredient, and preferably for the oral and/or transmucosal administration of an active ingredient.

12. Process of manufacture of a film delivery system comprising a mucoadhesive layer, comprising the steps of:
i) preparing a mucoadhesive layer; and
ii) providing the mucoadhesive layer with 5 to 100 perforations per cm² by using a perforating tool.

13. Process according to claim 12, wherein
in step ii), the mucoadhesive layer is provided with perforations by using a perforating tool selected from cutting devices, needle devices and punching devices, wherein preferably
the perforating tool is selected from needle devices selected from the group consisting of needles, needle assemblies, and pin rollers, and more preferably is provided with needles or pins having a diameter of at least 0.1, 0.2, 0.3 or 0.5 mm, or having a diameter of 3 mm or less, 2 mm or less, or 1 mm or less, or
the perforating tool is selected from punching devices, thereby removing the punched part from the mucoadhesive layer.

14. Film delivery system comprising a mucoadhesive layer, wherein
the mucoadhesive layer comprises a dissolvable film-forming agent and is provided with at least 10 to less than 20 perforations per cm²,
the perforations have a surface area per area of the mucoadhesive layer of at least 10% and 30% or less,
the perforations have a perimeter per area of the mucoadhesive layer of at least 4 cm/cm² and 15 cm/cm² or less,
the perforations all have a circular form and are distributed evenly, and
the mucoadhesive layer has an area weight of at least 200 g/m² and 400 g/m² or less.

15. Film delivery system comprising a mucoadhesive layer, wherein
the mucoadhesive layer comprises a dissolvable film-forming agent and is provided with at least 50 to less than 60 perforations per cm²,
the perforations have a surface area per area of the mucoadhesive layer of at least 5% and 30% or less,
the perforations have a perimeter per area of the mucoadhesive layer of at least 8 cm/cm² and 15 cm/cm² or less,
the perforations all have a circular form and are distributed evenly, and
the mucoadhesive layer has an area weight of at least 200 g/m² and 400 g/m² or less.
